(19) United States
(12) Patent Application Publication
(10) Pub. No.: US 2014/0296836 A1
(43) Pub. Date: Oct. 2, 2014

(54) GOLD-IN-SILICON NANOASSEMBLY FOR THERMAL THERAPY AND METHODS OF USE

(71) Applicants: BOARD OF REGENTS, THE UNIVERSITY OF TEXAS SYSTEM, Austin, TX (US); THE METHODIST HOSPITAL RESEARCH INSTITUTE, Houston, TX (US)

(72) Inventors: Haifa SHEN, Bellaire, TX (US); Mauro Ferrari, Houston, TX (US); Chun Li, Missouri City, TX (US); Jian You, Hangzhou (CN)

(73) Assignees: BOARD OF REGENTS, THE UNIVERSITY OF TEXAS SYSTEM, Austin, TX (US); THE METHODIST HOSPITAL RESEARCH INSTITUTE, Houston, TX (US)

(21) Appl. No.: 14/227,953

(22) Filed: Mar. 27, 2014

Related U.S. Application Data
(63) Continuation-in-part of application No. PCT/US2012/057365, filed on Sep. 26, 2012.
(60) Provisional application No. 61/539,285, filed on Sep. 27, 2011.

(51) Int. Cl. A61K 41/00; A61N 5/06; A61B 18/18; A61K 9/14; A61K 45/06

(52) U.S. Cl. CPC A61K 41/0052; A61K 9/143; A61K 45/06; A61B 18/18; A61N 5/0625; A61B 2018/1807; A61N 2005/0659
USPC 606/3; 424/489; 424/649; 424/9.3; 424/9.4; 424/9.1; 424/178.1; 424/94.1; 424/1.11; 607/100

(57) ABSTRACT

The present invention provides methods and compositions for the nanotechnology-based therapy of one or more mammalian diseases. Disclosed are gold-in-porous silicon nanoassemblies that are effective in the targeted and localized treatment of one or more human hyperproliferative disorders, including, for example, cancer of the breast. Methods of systemic administration of these nanoassembly vectors are disclosed that facilitate direct thermal ablative therapy of selected tissues using a localized application of near-infrared energy to the target site, wherein the gold-in-porous silicon nanoparticles release heat to destroy the surrounding cancerous tissue.

### CROSS-REFERENCE TO RELATED APPLICATIONS

The present application is a continuation-in-part of PCT Intl. Pat. Appl. No. PCT/US2012/057365, filed Sep. 26, 2012 (Atty. Dkt. No. 37182.142; pending), which claims priority to U.S. Pat. Appl. No. 61/539,285, filed Sep. 27, 2011; each of which is specifically incorporated herein in its entirety by express reference thereto.

### STATEMENT REGARDING FEDERALLY SPONSORED RESEARCH OR DEVELOPMENT

This invention was made with government support under Grant No. W81XWH-09-1-0212 awarded by the United States Department of Defense. The government has certain rights in the invention.

### BACKGROUND OF THE INVENTION

1. Field of the Invention

The present invention relates to the fields of biochemistry and medicine. In particular, the invention provides methods and compositions for thermal and/or ablative therapies of mammalian diseases, including, for example, mild heat to permeabilize tissue blood vessels for effective drug delivery, or the use of thermal ablation to threat one or more types of cancers. In illustrative embodiments, the invention provides multistage vector delivery systems that comprise biocompatible and biodegradable nanoparticle-loaded mesoporous silicon compounds particularly suited for the targeted thermal ablation of mammalian tumors, including in particular, human tumors such as those of the breast and other organs.

2. Description of Related Art

Despite decades of effort towards research and drug development aimed at treating cancer, the disease remains as one of the major causes of human death worldwide (Jemal et al., 2010). Human cancers typically consist of mixed populations of malignant cells, which may carry multiple genetic mutations. For example, an estimated forty DNA mutations may result in amino acid substitutions in an individual tumor of glioblastoma (Parsons et al., 2008) or pancreatic cancer (Jones et al., 2008). Nearly twice as many mutations have been demonstrated in certain breast and colorectal cancers (Wood et al., 2007); many of them are “driver” mutations—those that determine tumor initiation, progression and metastasis. The genomic landscapes indicate that multiple signal transduction pathways determine the fate of a cancer cell, and it is almost impossible to treat cancer with a single therapeutic agent.

It is not surprising, therefore, that many of the recently developed targeted therapy drugs (such as the EGFR inhibitors for non-small cell lung cancer (NSCLC) treatment and Herceptin® (trastuzumab, Genentech, South San Francisco, Calif., USA) for breast cancer therapy) are effective in only certain subsets of cancer patients (Lynch et al., 2004 and Paez et al., 2004). Unfortunately, in many cases, drug resistance may arise from additional genetic and epigenetic alterations. For example, it has been reported that mutations within the EGFR gene (Balak et al., 2006; Kobayashi et al., 2005), the KRAS gene (Pao et al., 2005; Eberhard et al., 2005), and up-regulation of other signaling pathways (Engelman et al., 2007; Bean et al., 2007) can both cause resistance to EGFR-targeted therapy. Clearly, there is an urgent and persistent motivation to develop new and useful therapeutic and/or diagnostic compositions—independent of cancer genetic background—for use in fighting this deadly disease.

Nanotechnology's Emerging Role in Cancer Therapy

Nanotechnology has played a crucial role in the development of cancer therapeutics (Ferrari, 2005). Doxil® (doxorubicin hydrochloride, Janssen Pharmaceuticals, Horsham, Pa., USA); and Abraxane® (paclitaxel protein-bound particles, Celgene Corp., Summit, N.J., USA) (Rahman, et al., 1990; Treat et al., 1990; Ibrahim et al., 2005; and Gradishar et al., 2005) are just two examples of nanoformulated drugs.

Gold Nanoparticles as Novel Agents for Cancer Therapy

Gold nanoparticles are currently being explored to induce hyperthermic cytotoxicity (Hirsch et al., 2003; Glazer and Curley, 2010; O'Neal et al., 2004; Schwartz et al., 2009; and Dickerson et al., 2008). When exposed to light at the right wavelength, the conduction-band electrons of the nanoparticle generate heat that is transmitted to the cells and surrounding tissues. Thermal therapy has the advantage of killing cancer cells without causing resistance—regardless of genetic background. Thus, it can be successfully applied to practically all cancer patients. However, successful application of this approach requires adequate accumulation of gold nanoparticles in the tumor and sufficient tumor penetration of the excitation energy (Kennedy et al., 2011). Due to lack of effective delivery, tumor delivery of gold nanoparticles in most studies has been relied on the enhanced permeability and retention (EPR) effect, a result of tumor blood vessel leakiness due to a state of ongoing angiogenesis, and thus are not very efficient (Maeda, 2001). It has been reported that less than 5% of total injected dose of PEGylated gold nanoparticles could ultimately reach the tumor tissue (Dickerson et al., 2008). The amount of gold nanoparticles needed for each treatment makes it impractical for clinic therapies. Besides, gold nanoparticles tend to accumulate unevenly in the tumor tissue dependent on particle size, surface charge, and other factors (Puvanakrishnan et al., 2009; Perrault et al., 2009; and Kim et al., 2010), which makes it difficult to eradicate the whole tumor tissue with this approach.

The inventors, their co-workers, and various scientific collaborators have previously developed a variety of micro-fabricated porous particles, and methods for using them in targeting cells. One such development is a multi-stage vector delivery system based on porous silicon (pSi) (see, e.g., U.S. Patent Appl. Nos. 2010/0074958, 2010/0029785, 2008/031182, 2008/0280140, 2008/0206344, 2008/0102030, 2003/0114366, each of which is specifically incorporated herein in its entirety by express reference thereto; various passages of which have been excerpted and/or reproduced herein).

Various nano- and micro-scale particles (also known as “nanovectors”) have also been developed in recent years for delivery of active agents, including such uses as vectors for delivery of one or more therapeutic or diagnostic (e.g., imaging) agents (see e.g., Ferrari, 2005). Illustrative examples of such nanovectors include silicon particles (see, e.g., Cohen et al., 2003), polymer-based particles (see, e.g., Duncan, 2003); quantum dots (see e.g., Alivisatos, 1996); iron oxide particles (see, e.g., Winter et al., 2003); gadolinium-containing particles (see e.g., Oyewumi and Mumper, 2004); gold nanoshells (see, e.g., Goldsmith and Turitto, 1986) and low-density lipid particulates (see, e.g., Bloch et al., 2004), to name only a few.

### Deficiencies in the Prior Art

Although thermal therapy with gold nanoparticles has been explored by multiple laboratories for many years, none, however, has yet yielded an effective therapeutic agent. Particular limitations encountered by these laboratories have including, inter alia, 1) unfavorable biodistribution of gold nanoparticles, 2) low accumulation of the gold nanoparticles in the targeted tissue of interest, and 3) low penetration of the near-infrared (NIR) energy source.

### BRIEF SUMMARY OF THE INVENTION

The present invention overcomes these and other limitations inherent in the prior art by providing inventive therapeutic and diagnostic nanoformulated vector compositions for use in the preparation of medicaments, and in methods for the diagnosis, treatment, and/or amelioration of one or more symptoms of mammalian disease. In particular, the invention provides new, non-obvious, and useful nanovector compositions suitable in the diagnosis, treatment, and/or amelioration of one or more symptoms of mammalian cancers, and in particular, human cancers, including those of the human breast, and other organs.

The porous silicon microparticles described herein can readily be tailored to target tumor tissues specifically, and they can deliver a sufficient quantity of nanoparticles into the targeted tissue to facilitate thermal ablation of the targeted cancer cells. Hollow gold nanoparticles have a favorable optimal absorbance at 800 nm, but when loaded into porous silicon, there is a red shift in absorbance by approximately 200 nm, which further increases tissue penetration dramatically.

Using the compositions described herein, methods for treating cancers, and for the thermal ablation of cancer cells (including, for example, solid tumors) have for the first time been facilitated using engineered multistage mesoporous silicon-based vectors. These vectors generate localized heat in response to NIR exposure.

In illustrative embodiments, the invention provides nanogold compositions useful in treating, and/or ameliorating at least one symptom of a disease, disorder, or dysfunction in an animal, and in particular, for the therapy and/or diagnostic imaging of cancers, and particularly mammalian tumors including, without limitation, cancers of the human breast.

The present invention provides effective nanotechnology-based therapeutic agents for use in the treatment of mammalian diseases and in particular, human diseases including cancer. The gold-in-porous-silicon nanoassembly composition provided herein generates heat efficiently when excited by an NIR energy source, such as a laser. When administered systemically to a subject in need thereof, the nanoassembly compositions localize preferably to one or more tissue(s) of interest or to one or more selected cell types), and, when subsequently energized by an externally-applied energy source, including, without limitation an infra-red, or more preferably an NIR energy source, produce sufficient localized thermal energy emission either to permeabilize blood vessels of a target tissue to allow efficient drug delivery, or to ablate one or more of the population of targeted cell(s) and/or one or more portions of the targeted tissue(s).

In a first embodiment, the invention provides a composition that includes at least one first stage particle that is a micro or nanoparticle and which has (i) a body, (ii) at least one surface; and (iii) at least one reservoir inside the body, such that the reservoir contains at least one second stage particle.

The first stage particle preferably has a substantially spherical, a substantially discoidal, a substantially hemispherical, a substantially cylindrical, or a substantially non-spherical shape, with an average size of the first-stage particle ranging substantially from about 600 to about 3500 nm in diameter, and more preferably from about 700 to about 3200 nm in diameter, and more preferably still, from about 800 nm to about 2600 nm in diameter, with all intermediate ranges and sizes being considered to explicitly fall within the scope of the present invention.

In particular applications, the body of the first stage particle substantially includes at least a first a porous or nanoporous silicon, with nanoporous silicon being particularly preferred. In the case of nanoporous silicon, preferably the pores within the material will be substantially within the range of about 30 nm to about 150 nm (nominal diameter). In certain aspects, the average pore size within the material will be on the order of from about 50 nm to about 130 nm, and more preferably, within the range of about 60 or 70 nm up to and including about 110 to 120 nm or so in nominal diameter size. Again, all intermediate ranges and sizes being considered to explicitly fall within the scope of the present invention.

Preferably, the second stage particle includes elemental (i.e., solid) gold, hollow gold, or a combination thereof. When gold is used as the second stage particle, the overall composition may be properly referred to as a “gold-in-silicon nanoassembly.”

Preferably the average size (i.e., the nominal particle diameter) of the second stage particle is substantially from about 5 nm to about 100 nm in diameter, more preferably from about 10 nm to about 90 nm in diameter, and more preferably still from about 20 nm or 30 nm to about 70 nm or 80 nm or so in diameter.

Optionally, the first stage particle may also further include one or more targeting or affinity moieties to facilitate directed or “targeted” delivery with one or more cell types, tissue types, surface receptors, binding domains, and such like. Such targeting or affinity moieties are preferably selected from the group consisting of a chemical targeting moiety, a physical targeting moiety, a geometrical targeting moiety and any combination thereof. Preferably, at least one targeting moiety is selected from the group consisting of a size of the body of the first stage particle; a shape of the body of the first stage particle; a charge on the surface of the first stage particle; a chemical modification of the first stage particle and any combination thereof.

In certain embodiments, the at least one targeting moiety includes at least one chemical targeting moiety disposed on or about the outer surface of the first-stage particle. Typically, the chemical targeting moiety will include at least one moiety such as a dendrimer, an aptamer, an antibody, an antigen binding fragment, a peptide, a thioaptamer, a protein, a ligand, a biomolecule, or any combination thereof.

In addition to the gold-in-silicon nanoassembly itself, the composition may further optionally include one or more additional therapeutic and/or diagnostic agents, and may include, for example, one or more additional therapeutic drugs, or one or more imaging agents, or any combination thereof. For example, the one or more additional agents may include at least one penetration enhancer, at least one additional active agent, at least one targeting moiety, or any combination thereof. In certain embodiments, a population of the first stage particles may be adapted and configured to retain a population of the second stage particles upon administration of the composition to the circulatory system of an animal. The population of first stage particles may also be adapted and configured to substantially prevent release of the population of second stage particles upon administration of the composition to the circulatory system of an animal.

In particular embodiments, the population of the first stage particles and the population of second stage particles form a thermal ablative nanoassembly, and in additional embodiments, the second stage particle further contains at least a first chemotherapeutic agent, and particularly one that is effective against one or more human cancer cells, and brain metastatic breast cancer cells in particular.

In additional embodiments, the second stage particle may further contain at least a first diagnostic compound, including one or more detection reagents, imaging agents, or any combination thereof.

The invention also provides a method of delivering a therapeutic or a diagnostic compound to at least a first cell, tissue, or organ within or about the body of an animal. In an overall and general sense the method includes administering to a subject an effective amount of a gold-in-silicon nanoassembly, for a time sufficient to provide the therapeutic or diagnostic compound to at least a first cell, tissue or organ of an animal in need thereof. In certain embodiments, the composition comprises a first-stage particle that is adapted and configured for localizing to the at least a first population of cells, tissues or an organ within or about the body of the animal (and preferably a mammal such as a human). In certain embodiments, the first cell is a cancer cell, a stem cell, a tumor cell, a clonogenic cell, or any combination thereof.

In the practice of the invention, the step of administering the composition may include providing the composition by one or more suitable modes of ordinary delivery of mammalian diagnostic or therapeutic agents, including, without limitation, by intravascular or systemic administration, or by subcutaneous delivery, or by a localized or a direct injection to one or more sites within or about the body.

The invention also provides a method for thermal ablation of a targeted cell or tissue in a mammal. This method generally involves administering to the mammal in need thereof, an effective amount of a pharmaceutical formulation that includes at least a first gold-in-silicon nanoassembly composition, and providing a sufficient amount of a NIR energy source in an amount and for a time sufficient to thermally ablate the targeted cell or tissue.

The invention also provides a method for mild thermal therapy of a targeted cell or tissue in a mammal. This method generally involves administering to the mammal in need thereof, an effective amount of a pharmaceutical formulation that includes at least a first gold-in-silicon nanoassembly composition, and providing a sufficient amount of a NIR energy source in an amount and for a time sufficient to heat the target tissue in order to permeabilize the blood vessels in order to facilitate drug entry into the tissue.

Preferably, the NIR source is a laser capable of emitting approximately over a wide range of discreet wavelengths, and in particular embodiments, energy at a wavelength of approximately 800-nm and/or 530-nm.

A method of treating or ameliorating one or more symptoms of cancer in an animal is also provided by the present invention. In an overall and general sense, the method generally includes at least the step of administering to a subject in need thereof, in the presence of a NIR energy source, an effective amount of a gold-in-silicon nanoassembly composition for a time sufficient to treat or ameliorate the one or more symptoms of at least a first cancer in the animal.

The disclosed pharmaceutical formulation may further include a buffer, a surfactant, a polymethacrylate, a biodegradable polymer, a biodegradable polyester, an aqueous polymeric gel, a microparticle, a nanoparticle, a liposome, a nanosphere, or any combination thereof. Preferably, the animal is mammalian, with humans being particularly preferred, especially those women diagnosed with breast cancer, including, for example, triple-negative breast cancer.

Preparation of Medicaments

Another important aspect of the present invention concerns methods for using the disclosed multistage vectors (as well as compositions or formulations including them) in the preparation of medicaments for treating or ameliorating the symptoms of one or more diseases, dysfunctions, or deficiencies in an animal, such as a vertebrate mammal. Use of the disclosed nanoparticle gold-in-silicon compositions is also contemplated in therapy and/or treatment of one or more diseases, disorders, dysfunctions, conditions, disabilities, deformities, or deficiencies, and any symptoms thereof.

Such use generally involves administration to an animal in need thereof one or more of the disclosed compositions, either alone, or further in combination with one or more additional therapeutic agents, in an amount and for a time sufficient to treat, lessen, or ameliorate one or more of a disease, disorder, dysfunction, condition, disability, deformity, or deficiency in the affected animal, or one or more symptoms thereof, including, without limitation one or more tumors, such as those of the mammalian breast.

Compositions including one or more of the disclosed pharmaceutical formulations also form part of the present invention, and particularly those compositions that further include at least a first pharmaceutically acceptable excipient for use in the therapy and/or imaging of one or more diseases, dysfunctions, disorders, or such like, including, without limitation, one or more cancers or tumors of the human body.

Use of the disclosed compositions is also contemplated, particularly in the manufacture of medicaments and methods involving one or more therapeutic (including chemotherapy, phototherapy, laser therapy, etc.) prophylactic (including e.g., vaccines), or diagnostic regimens, (including, without limitation, in diagnostic imaging, such as CT, MRI, PET, ultrasonography, or the like).

The pharmaceutical formulations of the present invention may optionally further include one or more additional distinct active ingredients, detection reagents, vehicles, additives, adjuvants, therapeutic agents, radionuclides, gases, or fluorescent labels as may be suitable for administration to an animal. Such routes of administration are known to and may be selected by those of ordinary skill in the art, and include, without limitation, delivery devices including intramuscular, intravenous, intra-arterial, intrathecal, intracavitary, intraventricular, subcutaneous, or direct injection into an organ, tissue site, or population of cells in the recipient animal.

The use of one or more of the disclosed compositions in the manufacture of a medicament for therapy of one or more mammalian cancer is also an important aspect of the invention. Formulation of such compositions for use in administration to an animal host cell, and to a mammalian host cell in particular, is also provided by the invention. In particular embodiments, the invention provides for formulation of such compositions for use in administration to a human, or to one or more selected human host cells, tissues, organs in situ, or to an in vitro or ex situ culture thereof, for the purpose of localized thermal ablation of cells or tissues within or about the body of the animal, with uses for the thermal ablation of cancer cells being particularly preferred.

The present invention also provides for the use of one or more of the disclosed microporous silicon-vector gold nanoparticle compositions in the manufacture of a medicament for the treatment of one or more mammalian cancers, including the preparation of one or more therapeutic regimens for the treatment or ameliorate of one or more symptoms of mammalian tumors such as triple-negative human breast tumors.

The invention also provides methods for providing a therapeutic amount of a nanoparticle-enabled thermal ablative agent to a population of cells or to one or more tissues within the body of a mammal, with the method generally including providing to a mammal in need thereof an effective amount of a therapeutic composition as disclosed herein for a time effective to provide the desired therapy in the selected cells or tissue targeted within the mammal to undergo thermal ablation.

The pharmaceutical compositions of the present invention may be administered to a selected animal using any of a number of conventional methodologies, including, without limitation, one or more of parenteral, intravenous, intraperitoneal, subcutaneous, transcutaneous, intradermal, subdermal, transdermal, intramuscular, topical, intranasal, insertion, inhalation, insufflation, ingestion, or by localized or direct administration to one or more sites within or about the body of the animal.

Yet another advantage of the present invention may include active ingredient(s) and pharmaceutical formulations and compositions that include one or more of such active ingredients useful in treating or ameliorating one or more symptom(s) of an infection, disease, disorder, dysfunction, trauma, or abnormality in a mammal. Such methods generally involve administration to a mammal, and in particular, to a human, in need thereof, one or more of the pharmaceutical compositions, in an amount and for a time sufficient to treat, ameliorate, or lessen the severity, duration, or extent of, such a disease, infection, disorder, dysfunction, trauma, or abnormality in such a mammal.

As described herein, the disclosed pharmaceutical compositions may also be formulated for diagnostic and/or therapeutic uses, including their incorporation into one or more diagnostic or therapeutic kits packaged for clinical, diagnostic, and/or commercial resale. The compositions disclosed herein may further optionally include one or more detection reagents, one or more additional diagnostic reagents, one or more control reagents, one or more targeting reagents, ligands, binding domains, or such like, and/or one or more therapeutic or imaging compounds, including, without limitation, radionuclides, fluorescent moieties, and such like, or any combination thereof. In the case of diagnostic reagents, the compositions may further optionally include one or more detectable labels that may be used in both in vitro and/or in vivo diagnostic, therapeutic, and/or prophylactic modalities.

Active Agents

In preferred embodiments, the nano-gold particles contained within the microporous silicon particles act as an active therapeutic agent per se. The nano-gold particles generate heat when exposed to light. Mild hyperthermia (42-44° C.) can not only permeabilize tumor blood vessels in order to improve the transport of macromolecular drugs and nanoparticles (see, Kirui et al., 2014), but also kill cancer stem cells (Atkinson et al., 2010). In the even higher temperature range, thermal ablation has the potential to kill every cell type.

In other embodiments, the multistage vectors of the invention may be combined with a further therapeutic compound or an imaging agent, to provided enhanced therapy and/or diagnosis. Examples of conventional active agents that may be coadministered with the thermal-ablative vectors of the invention may include, for example, one or more peptides, proteins, nucleic acids, and/or small molecules. Such therapeutic agents may be in various forms, such as an unchanged molecule, molecular complex, pharmacologically acceptable salt, such as hydrochloride, hydrobromide, sulfate, laurate, palmitate, phosphate, nitrite, nitrate, borate, acetate, maleate, tartrate, oleate, salicylate, and the like. For acidic therapeutic agent, salts of metals, amines or organic cations, for example, quaternary ammonium can be used. Derivatives of drugs, such as bases, esters and amides also can be used as a therapeutic agent. A therapeutic agent that is water insoluble can be used in a form that is a water soluble derivative thereof, or as a base derivative thereof, which in either instance, or by its delivery, is converted by enzymes, hydrolyzed by the body pH, or by other metabolic processes to the original therapeutically active form.

In particular embodiments, the therapeutic agent can be a chemotherapeutic agent, an immunosuppressive agent, a cytokine, a cytotoxic agent, a nucleolytic compound, a radioactive isotope, a receptor, an antibody-small molecule conjugate, or a pro-drug activating enzyme. The delivered agent may be naturally occurring, produced by synthetic and/or recombinant methods, or any combination thereof.

Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) can have particular utility as the therapeutic agent.

In the practice of the invention, the delivery of one or more anti-cancer agents, and cancer chemotherapy agents is a particularly preferred therapeutic use of the disclosed delivery vectors. Useful cancer chemotherapy drugs include, without limitation, nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors, hormonal agents, and one or more combinations thereof. Other anti-cancer agents include small inhibitory ribonucleic acids (siRNA), small hairpin ribonucleic acids (shRNA), and micro-ribonucleic acids (miRNA) that inhibit expression of genes that play key roles on tumor initiation, promotion, progression, and metastasis.

Exemplary chemotherapy drugs include, without limitation, actinomycin-D, alkeran, Ara-C, anastrozole, asparaginase, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, cacarbazine, cactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, herceptin, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, rituximab, steroids, streptozocin, STI-571, streptozocin, tamoxifen, temozolomide, teniposide, tetrazine, thioguanine, thiotepa, tomudex, topotecan, treosulphan, trimetrexate, vinblastine, vincristine, vindesine, vinorelbine, VP-16, Xeloda, and one or more combinations thereof.

Useful cancer chemotherapy drugs suitable for delivery to one or more cancer cells, tissues, tumors, or any combination thereof using the vectors of the present invention also include, without limitation, one or more alkylating agents (e.g., thiotepa and cyclosphosphamide); alkyl sulfonates (e.g., busulfan, improsulfan and piposulfan); aziridines (e.g., benzodopa, carboquone, meturedopa, and uredopa); ethylenimines and methylamelamines (including, without limitation, altretamine, triethylenemelamine, trietylenephosphoramide, triethylenethiophosphaoramide and trimethylolomelamine); nitrogen mustards such as chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, and uracil mustard; nitroureas (including, without limitation, cannustine, chlorozotocin, fotemustine, lomustine, nimustine, and ranimustine; antibiotics such as aclacinomysins, actinomycin, authramycin, azaserine, bleomycins, cactinomycin, calicheamicin, carabicin, caminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin); anti-metabolites [e.g., 5-fluorouracil (5-FU)]; folic acid analogs including, without limitation, denopterin, pteropterin, and trimetrexate; purine analogs including, without limitation, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, rnepitiostane, and testolactone; anti-adrenals such as aminoglutethimide, mitotane, and trilostane; folic acid replenishers (including, without limitation, frolinic acid); aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofrran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotEPa; taxoids [including, e.g., paclitaxel (TAXOL®, Bristol-Myers Squibb Oncology, Princeton, N.J., USA) and doxetaxel (TAXOTERE®, Rhone-Poulenc Rorer, Antony, France)]; chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vinorelbine; navelbine; novantrone; daunomycin; xeloda; CPT-11; difluoromethylornithine (DMFO); retinoic acid; esperamicins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4 hydroxytamoxifen, rrioxifene, keoxifene, onapristone, and toremifene (Fareston); and anti-androgens such as flutamide, nilutamide, bicalutamide, leuprolide, and goserelin; and pharmaceutically acceptable salts, acids or derivatives of any of the above.

The diagnostic and/or imaging agents that can be delivered by the compounds of the present invention may include, without limitation, any substance that can provide diagnostic or imaging information about one or more selected site without or about the body of an animal (and of a human, in particular). Such diagnostic and/or imaging agents can include one or more magnetic materials (such as iron oxide), for use in one or more magnetic resonance imaging-based modalities. For use in optical imaging, the active agent can include, for example, one or more semiconductor nanocrystals or quantum “dots.” For use in optical coherence tomographic imaging, the active agent may include one or more metals (e.g., gold or silver. The imaging agents for use in the present invention may also include one or more ultrasound contrast agents (including, without limitation, microbubbles, nanobubbles, or iron oxide micro- or nano-particle based compositions) or any combination thereof.

The application of the compositions and methods of the present invention for use in one or more of the various commercially-available diagnostic and/or imaging modalities presently in use in the medical arts will be apparent to the person of ordinary skill in the art having benefit of the teachings of the present invention, and are therefore, not further elaborated herein.

Pharmaceutical Compositions

The drug delivery vehicles of the present invention are preferably formulated into one or more pharmaceutical compositions suitable for administration to an animal, and to a mammal such as a human in particular. Such compositions can be a suspension that includes one or more of the delivery agents described herein, and may find particular utility in delivering or facilitating administration of one or more therapeutic or diagnostic compounds to one or more biological cells, tissues, organs, or one or more regions of interest within, or about, the body of an animal.

The methods of the present invention are particularly useful in improving patient outcomes over currently practiced therapies by more effectively providing an effective amount of thermal ablative therapy to populations of cells or one or more tissue sites within the body of an animal. In certain circumstances, the present invention may diminish unwanted side effects of conventional therapy.

Preferably, the compounds of the present invention will generally be formulated for systemic and/or localized administration to an animal, or to one or more cells or tissues thereof, and in particular, will be formulated for systemic and/or localized administration to a mammal, or to one or more cells or tissues thereof. In certain embodiments, the compounds and methods disclosed herein will find particular use in the localized thermal ablation of one or more targeted cells or tissues, such as a tumor, within or about the body of a mammal, and preferably, a human being.

The present invention also provides for the use of one or more of the disclosed pharmaceutical compositions in the manufacture of a medicament for therapy, and particularly for use in the manufacture of a medicament for treating, and/or ameliorating one or more symptoms of a disease, dysfunction, or disorder in a mammal, and in a human in particular.

The present invention also provides for the use of one or more of the disclosed pharmaceutical compositions in the manufacture of a medicament for therapy or amelioration of symptoms of one or more medical conditions such as hyperproliferative disorder, cancer, and/or tumors in a mammal.

In certain embodiments, the present invention concerns formulation of one or more therapeutic or diagnostic agents in a pharmaceutically acceptable composition for administration to a cell or an animal, either alone, or in combination with one or more other modalities of prophylaxis and/or therapy. The formulation of pharmaceutically acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the stress-inducible targeted drug delivery compositions disclosed herein in suitably-formulated pharmaceutical vehicles by one or more standard delivery devices, including, without limitation, subcutaneously, intraocularly, intravitreally, parenterally, intravenously, intracerebroventricularly, intramuscularly, intrathecally, orally, intraperitoneally, transdermally, topically, by oral or nasal inhalation, or by direct injection to one or more cells, tissues, or organs. The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of ordinary skill in the art in light of the present disclosure. For example, one dosage may be dissolved in 1 mL of isotonic NaCl solution, and either added to 1000 mL of hypodermoclysis fluid or injected at the proposed site of infusion, (see, e.g., “Remington's Pharmaceutical Sciences” 15th Edition, pages 1035-1038 and 1570-1580). Some variation in dosage will necessarily occur depending on the condition of the subject being treated. The person responsible for administration will determine, in any event, the appropriate dose for the individual subject. Moreover, for human administration, preparations should meet sterility, pyrogenicity, and the general safety and purity standards as required by FDA Office of Biologics standards.

Sterile injectable compositions may be prepared by incorporating the disclosed drug delivery vehicles in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form. Pharmaceutically-acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of the compositions disclosed herein will be within the purview of the ordinary-skilled artisan having benefit of the present teaching. It is likely, however, that the administration of a therapeutically-effective, pharmaceutically-effective, prophylactically-effective, or diagnostically-effective amount of the disclosed pharmaceutical compositions may be achieved by a single administration, such as, without limitation, a single injection of a sufficient quantity of the delivered agent to provide the desired benefit to the patient undergoing such a procedure. Alternatively, in some circumstances, it may be desirable to provide multiple, or successive administrations of the stress-inducible targeted drug delivery compositions, either over a relatively short, or even a relatively prolonged period of time, as may be determined by the medical practitioner overseeing the administration of such compositions to the selected individual.

Typically, formulations of one or more active ingredients in the drug delivery formulations disclosed herein will contain an effective amount for the selected therapy or diagnosis. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80 weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological t_{1/2}, route of administration, product shelf life, as well as other pharmacological considerations will be contemplated by one of ordinary skill in the art of preparing such pharmaceutical formulations, and as such, a variety of dosages and treatment regimens may be desirable.

The pharmaceutical compositions disclosed herein may be administered by any effective method, including, without limitation, by parenteral, intravenous, intramuscular, or even intraperitoneal administration as described, for example, in U.S. Pat. Nos. 5,543,158, 5,641,515 and 5,399,363, each of which is specifically incorporated herein in its entirety by express reference thereto. Solutions of the gold-in-silicon nanoassembly compositions (either alone, or in combination with one or more additional active compounds) prepared free-base or in one or more pharmacologically acceptable buffer or salt solutions may be mixed with one or more surfactants, such as e.g., hydroxypropylcellulose, depending upon the particular application. The pharmaceutical forms adapted for injectable administration of the disclosed gold-in-silicon nanoassembly compositions include, without limitation, sterile aqueous solutions or dispersions, as well as one or more sterile powders for the extemporaneous preparation of sterile injectable solutions or dispersions including without limitation those described in U.S. Pat. No. 5,466,468 (which is specifically incorporated herein in its entirety by express reference thereto). In most applications of the invention, the pharmaceutical formulation preferably be a sterile solution, and one that is sufficiently fluid to the extent that easy syringability of the formulation exists. Such formulations are also preferably sufficiently stable under the conditions of normal manufacture, storage, and transport, and are preferably preserved against the contaminating action of one or more microorganisms, such as viruses, bacteria, fungi, yeast, and such like.

The carrier(s) or vehicle(s) employed for delivery of the nanoassembly compositions may be any conventional pharmaceutical solvent and/or dispersion medium including, without limitation, water, alcohols such as ethanol, polyols such as glycerol, propylene glycol, and liquid polyethylene glycol, and the like, or a combination thereof), one or more vegetable oils, or any combination thereof. Additional pharmaceutically-acceptable components may be included, such as to improve fluidity, prevent dissolution of the reagents, and such like.

Proper fluidity of the pharmaceutical formulations disclosed herein may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, without limitation, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

Systemic administration of the compounds of the present invention is particularly contemplated to be an effective mode of providing the nanoassembly compositions, and any optional additional therapeutic or diagnostic agents that may be included within a formulation of such nanoassemblies. However, in many embodiments of the invention, it is also contemplated that formulations of the disclosed nanoassembly compositions may be suitable for direct injection into one or more organs, tissues, or cell types in the body. Such injection sites include, without limitation, the circulatory system, the spinal cord, the lymphatic system, a joint or joint capsule, a synovium or subsynovium tissue, tendons, ligaments, cartilages, bone, periarticular muscle or an articular space of a mammalian joint, as well as direct administration to an organ or tissue site such as the breast, heart, liver, lung, pancreas, intestine, brain, bladder, kidney, or other site within the patient's body, including, for example, without limitation, introduction of the delivered therapeutic or diagnostic agent(s) via intra-abdominal, intra-thoracic, intravascular, or intracerebroventricular delivery of a suitable liposomal formulation. In particular, the inventors contemplate the direct application of the formulations disclosed herein to one or more solid tumors or to one or more cancerous tissues or organs within, or about the body of a human undergoing treatment for a cancer such as breast cancer.

Administration of the disclosed nanoassembly compositions need not be restricted to one or more of these particular delivery modes, but in fact, the compositions may be delivered using any suitable delivery mechanism, including, for example, those known to the one of ordinary skill in the pharmaceutical and/or medical arts. In certain embodiments, the active ingredients of the invention may be formulated for delivery by needle, catheter, and related means, or alternatively, may be included within a medical device, including, without limitation, a drug-eluting implant, a catheter, or any such like device that may be useful in directing the compositions to the selected target site in the animal undergoing treatment.

The administration of the gold-in-silicon nanoassembly compositions disclosed herein may be conducted using any method as conventionally employed in the medical arts, and may include, without limitation, administration of intranasal sprays, inhalation, and/or other aerosol delivery vehicles (see e.g., U.S. Pat. Nos. 5,756,353 and 5,804,212, each of which is specifically incorporated herein in its entirety by express reference thereto). Delivery of drugs using intranasal microparticle resins (see e,g., Takenaga et al., 1998) and lysophosphatidyl-glycerol compounds (U.S. Pat. No. 5,725,871, specifically incorporated herein in its entirety by express reference thereto) are also well-known to those of ordinary skill in the pharmaceutical arts, and may also be employed in the practice of the present methods. Transmucosal drug delivery is also contemplated to be useful in the practice of the invention. Exemplary methods are described, for example, without limitation, in U.S. Pat. No. 5,780,045 (specifically incorporated herein in its entirety by express reference thereto).

The pharmaceutical formulations disclosed herein are not in any way limited to use only in humans, or even to primates, or mammals. In certain embodiments, the methods and compositions disclosed herein may be employed using avian, amphibian, reptilian, or other animal species. In preferred embodiments, however, the compositions of the present invention are preferably formulated for administration to a mammal, and in particular, to humans, in a variety of therapeutic, and/or diagnostic regimens. The compositions disclosed herein may also be provided in formulations that are acceptable for veterinary administration, including, without limitation, to selected livestock, exotic or domesticated animals, companion animals (including pets and such like), non-human primates, as well as zoological or otherwise captive specimens, and such like.

### BRIEF DESCRIPTION OF THE DRAWINGS

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1A, FIG. 1B, FIG. 1C, and FIG. 1D show scanning electron microscope (SEM) images of empty pSi and pSi/HAuNS. The SEM imaging of particles was performed using a ZEISS NEON 40 scanning electron microscope. To prepare SEM sample, a drop of IPA particle suspension was directly placed on a clean aluminum SEM sample stub and dried. The samples were loaded in SEM chamber, and SEM images were measured at 5 kV and 3-5 mm working distance using an in-lens detector. (FIG. 1A and FIG. 1B): SEM images of mono-dispersed 1000 nm×400 nm discoidal pSi particles with 60 nm mean pore size. (FIG. 1C): SEM image of silicon particles loaded with HAuNS. (FIG. 1D): Absorption spectra of pSi (purple), HAuNS (red), and pSi/HAuNS (blue);

FIG. 2 shows the heat generation kinetics from free HAuNS and pSi/HAuNS. Temperature change was measured over a period of 10 min of exposure to NIR with a wavelength of 808 nm, and an output power of 0.5 W. The same amount of HAuNS particles were used in the samples of free HAuNS and pSi/HAuNS. An equal amount of unloaded pSi particles was used as a control. Experimental data are shown with best exponential fit;

FIG. 3A, FIG. 3B, FIG. 3C, FIG. 3D, FIG. 3E, FIG. 3F, FIG. 3G and FIG. 3H show the photothermal effect on cancer cell growth in vitro and in vivo. Cell survival is plotted in FIG. 3A, FIG. 3B, and FIG. 3C as a function of HAuNS concentration. Cancer cells were incubated with a high dose (2×10¹⁰/well, in blue) and a low dose (2×10⁹/well, in purple) of free AuNP, free HAuNS, or pSi/HAuNS, and treated with NIR. Cell survival was measured by the MTT assay. Percentage of cell growth was calculated by comparing the growth of treated cells to untreated control cells. FIG. 3A: MDA-MB-231 cells; FIG. 3B: SK-BR-3 cells; and FIG. 3C: 4T1 cells. FIG. 3D-FIG. 3G show the cell viability staining after NIR treatment of MDA-MB-231 cells incubated with free HAuNS or pSi/HAuNS. MDA-MB-231 cells were incubated with a low-dose (1×) or a high dose (4×) free HAuNS or pSi/HAuNS, and treated with NIR. Live cells were stained green with calcein AM, and dead cells were stained red with EthD-1. The boundary of NIR laser beam was marked with a white line in each well, and the area hit by laser was marked with an asterisk. FIG. 3H shows the analysis of cell staining result. Percentage of dead cells in each well was normalized with untreated control cells;

FIG. 4 shows the photothermal therapy of murine 4T1 tumor. Mice were inoculated with 4T1 tumor cells, and divided into 4 treatment groups (n=8). When tumors reached an average size of 150-200 cm³, the tumor mice were administrated with free HAuNS or the same amount of HAuNS in pSi nanoassembly by intra-tumor injection, and treated with NIR. The PBS and pSi groups served as controls. The result was a summary of tumor weight 10 days after treatment;

FIG. 5A, FIG. 5B, and FIG. 5C show a schematic illustration of the overall aspects of one or more multistage vector platforms provided by the present invention.

FIG. 5A shows a non-toxic biodegradable first-stage carrier (grey) is optimally designed to evade RES and have margination, adhesion, internalization properties to attain preferential concentration on the target tumor vascular endothelium. In FIG. 5B, the first-stage particle co-releases second-stage carrier nanoparticles at the tumor vasculature. In FIG. 5C, the second-stage nanoparticles are shown penetrating the cellular membrane, from which they can then deploy different, synergistic therapeutic agents into the cytoplasm, the nucleus, or other subcellular targets as desired;

FIG. 6 shows the absorption spectra of gold nanoparticles in colloid suspension and pSi. Red: free AuNP; purple: empty pSi; blue: pSi/AuNP;

FIG. 7A, FIG. 7B, FIG. 7C, FIG. 7D, and FIG. 7E show cell viability after NIR treatment of MDA-MB-231 cells incubated with free HAuNS (FIG. 7A, FIG. 7B, and FIG. 7C) or pSi/HAuNS (FIG. 7D and FIG. 7E). The boundary of NIR laser beam was marked with a white line in each well, and the area hit by laser was marked with an asterisk. Percentage of dead cells in each well was normalized with untreated control cells;

FIG. 8 shows SK-BR-3 cell survival as a function of HAuNS concentration. Cancer cells were incubated with different doses of free HAuNS or pSi/HAuNS, and treated with NIR. Cell survival was measured by the MTT assay. Percentage of cell growth was calculated by comparing the growth of cells treated with HAuNS to cells without HAuNS;

FIG. 9 shows the enhanced heat generation of MSV/HAuNS triggered by NIR energy. In this study, a quartz cuvette was filled with equal amounts of free HAuNS or HAuNS-loaded in an exemplary microporous silicon vector of the invention (MSV/HAuNS) suspended in water. A continuous-wave GCSLX-01-1600m-1 fiber-coupled diode laser with a center wavelength of 808 was used to trigger heat generation. The output power was 1 Watt (W) for a spot diameter of 3.5 mm. A thermocouple was inserted into the solution perpendicular to the path of the laser light to record the change of temperature. Free silicon particles (MSV) did not contribute to heat generation. Heat was generated from free HAuNS triggered by NIR excitation. However, MSV/HAuNS was much more efficient in heat generation as demonstrated by both the slope of the initial curve, and the overall temperature obtained over the 10-min course of temperature measurement;

FIG. 10 shows the schematic representation of covalent ligation and formation of MSVs-gold NRs. The strategy forms a robust assembly coating for uses for long-term delivery applications. It is a one-pot assembly process which can be carried out at pH=8.5.

FIG. 11A, FIG. 11B, and FIG. 11C show enhanced heat conversion efficacy of MSVs after coating with gold nanostructures. FIG. 11A: Scanning microscopy micrograph showing porous MSVs; FIG. 11B: MSVs coated with gold nanorods increased absorbance in the near infrared; and FIG. 11C: Increased near infrared absorbance MSVs-gold nanorods which translate to enhancement of energy-absorbing capabilities coating with gold;

FIG. 12A, FIG. 12B, and FIG. 12C show the creation of MSVs-NRs hybrid particles with high-energy conversion efficiency as an attractive clinical therapeutic. FIG. 12A shows the heating profile of MSVs-NRs. A solution containing MSVs-NRs heated up rapidly when compared to the heating of a saline control solution. FIG. 12B shows a histological tissue sample of pancreatic cancer with intact cells and morphology whereas FIG. 12C shows the same cancer tissue after treatment with MSVs-NRs solution. Thermal destruction of the cancer cells was apparent, with the tissue sample showing extensive tissue damage and morphological corruption caused by the localized heat that was induced from NIR excitation of the MSVs-NRs;

FIG. 13A and FIG. 13B show nanoparticle-assisted mild hyperthermia (MHT) treatment enhances on tumor perfusion and permeability. (FIG. 13A) Graph showing relative perfusion change as function of time, where perfusion rates quickly rise 11-fold compared to initial baseline and 3-fold higher after cessation; (FIG. 13B) Amounts of Evans blue dye in tumor homogenate 1 and 3 hrs after hyperthermia as quantified fluorometrically at 470/680 nm excitation/emission wavelengths, respectively. Both time points demonstrated a significant effect in dye extravasation over the unheated control (2-fold increase at 1 hr and 2.5 fold increase at 3 hrs) with statistical significance denoted by *p<0.02, ** p<0.004 (n=5); and

FIG. 14A and FIG. 14B show mild hyperthermia (MHT) treatment enhances macromolecule extravasation up to 24 hrs which abates at 36 hrs after treatment; (FIG. 14A) (top panel) Intravital microscopic images show enhanced dye extravasation (red) in interstitial space at 5 hr after MHT and circulation of 2,000-kDa dextran dye; (bottom panel) show reduced dye accumulation in unheated control with majority of dye in vascular region with little in interstitial space; FITC dextran dye was injected immediately (˜1 min) and used as vessel tracer; FIG. 14B) Total amount of 2,000-kDa dextran in tumor homogenate after MHT treatment and quantified by spectrofluorimetrically at 561/595 nm emission/excitation wavelengths. Statistical significance is denoted by *p<0.046, **p<0.004, ***p<0.002.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

In the present invention, the delivery of hollow gold nanoshells (HAuNS) with MSV for thermal ablation therapy of triple negative breast cancer (TNBC) has been demonstrated.

The vector systems of the present invention are typically comprised of two delivery carriers: the first stage is a biodegradable pSi vector, and the second stage nanoparticles loaded into the pores of pSi. The incorporated nanoparticles can include liposomes or micelles that contain one or more therapeutic and/or diagnostic agents (i.e., a “third-stage” particle) (see Tasciotti, 2008). A major advantage of this system is that the size, shape, and surface properties of pSi can be precisely controlled, so that maximal tissue-specific localization and release of the delivered therapeutic(s) and/or diagnostic(s) at the target tumor can be achieved (Tasciotti, 2008; Ferrari, 2010; Decuzzi and Ferrari, 2008; and Decuzzi et al., 2010). The inventors and their co-workers also recently demonstrated successful application of this system to deliver siRNA therapeutics for cancer treatment with two mouse models of ovarian cancers (Tanaka et al., 2010; and Ferrari, 2010).

In an important aspect of the invention, a multistage vector (MSV) delivery system is provided that generally includes biocompatible and biodegradable mesoporous silicon particles and nanoparticles loaded into the pores of the silicon. This system offers tissue-specific delivery of nanoformulated therapeutics and diagnostic imaging agents for personalized therapy of human diseases. These MSVs, when delivered systemically, can travel throughout the circulatory system, and settle at tumor vasculature where the nanoparticles are then released from the mesoporous silicon, and subsequently enter the tumor tissue. The inventors and their collaborators previously demonstrated that multistage delivery of liposomal EphA2 siRNA provided sustained gene silencing, and inhibited ovarian tumor growth in a mouse orthotopic tumor model with a single dose of nanotherapeutics (Tanaka et al., 2010). TNBC cells are ER- and PR-negative, and lack overexpression of HER2. This type of cancer accounts for 12-20% of all breast cancer cases. Although TNBC tumors do respond to conventional chemotherapy, their relapse rate remains high. The overall poor prognosis, coupled with the high tendency for relapse both demand new therapeutic options for TNBC. Tumor accumulation of various mesoporous silicon particles with variable parameters including size, shape, and surface chemical modifications was examined in mouse models of human breast cancers, from which it was shown that 1,000×400 nm discoidal particles had a significantly high tumor accumulation rate. HAuNS with an average diameter of 30 nm could be efficiently loaded into the pores of MSV. NIR was used to trigger heat generation by the HAuNS. Surprisingly, the MSV-loaded HAuNS were much more efficient in heat generation than the same amount of free HAuNS in solution, a result that was most likely due to the confinement of the small HAuNS in the pores of the pSi particles.

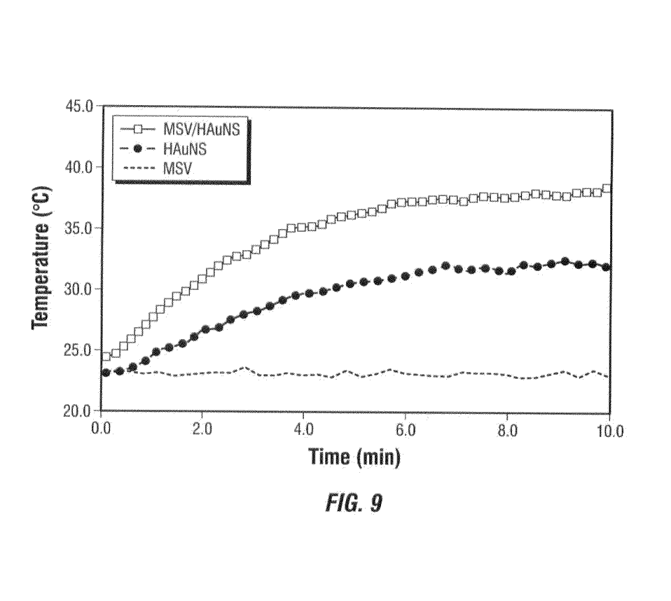

In cell culture, MSVs were efficiently taken up by human TNBC MDA-MB-231 cells. NIR treatment of these cells resulted in three times as many dead cells in the MSV group as in the free HAuNS group. Mice bearing MDA-MB-231 tumors were treated with free HAuNS or MSV/HAuNS, and tumors were exposed to NIR. Both free HAuNS and MSV/HAuNS were effective in inhibiting tumor growth; however, the inhibition was more profound in the MSV/HAuNS group than in the free HAuNS group (FIG. 9).

Gold-in-Silicon Nanoassemblies

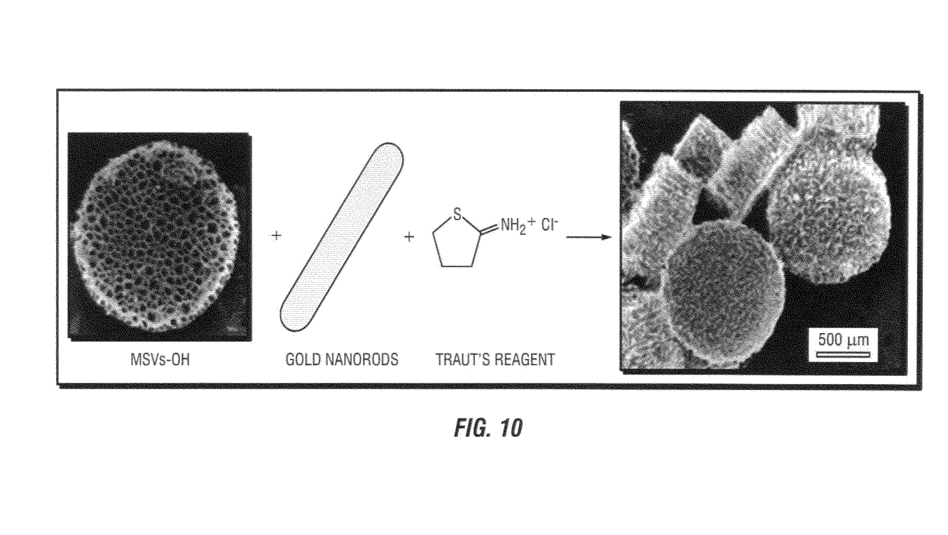

An exemplary gold-in-silicon nanoassembly of the present invention (see e.g., FIG. 9 and FIG. 10) includes a population of porous silicon (pSi) particles loaded with populations of gold nanoparticles. The porous silicon particles of the invention may be substantially of any shape (such as, e.g., and without limitation, cylindrical, discoidal, hemispherical, spherical), any size (preferably its average diameter ranges between about 600 nm and about 3500 nm), may have a variety of surface properties (e.g., unmodified, chemically modified, positively charged, negatively charged, etc.). If desired, the surface of the first-stage mesoporous silicon particles can be conjugated with one or more affinity moieties (e.g., peptides, antibodies, aptamers, thioaptamers, and the like) for tissue-specific targeting. The average size of the pores inside the porous silicon can vary within a wide range (e.g., from about 30 to about 150 nm or more), provided the gold or hollow gold nanoparticles can enter the pore space). Exemplary second-stage gold nanoparticles may be formed from solid gold, or alternatively, hollow gold, and may have an average particle size of from about 5 to about 100 nm in diameter. To fabricate the nanoassembly, second-stage gold nanoparticles are added into the first-stage porous silicon particles, with the gold nanoparticles entering the pores of the porous silicon by a combination of capillary force and surface interaction between gold nanoparticle and porous silicon (such as electric charge).

For the treatment of diseases such as mammalian cancers, these nanoassemblies may be delivered to the target cells or tissues either systemically (e.g., by i.v. or s.c. injection), or, if the target cells or tissues are either on the surface of the body, or reachable (e.g., through surgical intervention), the nanoassembly can also be directly administered into the target cells or cancer tissues. After the nanoassembly has reached to the selected therapy site within or about the body of the animal, a near infrared laser (e.g., preferably having an about 800-nm wavelength for hollow gold nanoparticles, or an about 530-nm wavelength for solid gold nanoparticles) may be used to energize the nanoassembly.

The resulting localized heat that is generated from the energized nanoassembly then provides the destructive thermal ablative energy necessary to destroy the target tissue, and to kill the population of cancer cells present. Preferably, the nanoassembly remains substantially intact, because studies have demonstrated that the ablative properties of the nanoassembly are most effective when the second-stage gold nanoparticles remain substantially within the porous silicon microparticles. Separation of the second stage gold nanoparticles from the first stage porous silicon microparticles has been shown to significantly reduce the ablative efficiency of thermal therapy vectors.

Several studies have been performed to determine heat generation from the gold-in-porous silicon nanoassembly. Surprisingly, the inventors have shown that heat was generated from the nanoassembly much more quickly and efficiently, and this thermal ablative capability has been exploited to kill cancer cells by this nanoassembly in a manner that was much more efficient than when using the same amount of free gold nanoparticles (whether in cell culture, or within a tumor tissue inside the body). Using the gold nanoparticle-loaded pSi particles described herein, the efficiency of photothermal therapy is dramatically improved.

Based upon these results, the inventors have also identified a possible mechanism for the surprising, and synergistic combination of thermal properties of the gold nanoparticles and the nanoscale organization of the pSi host material.

Nano-gold particles within the confinement of the nanoassembly become electromagnetically coupled through dipole-dipole interactions, and can function as waveguide-like structures that increase energy transfer and heat production. As a result, all nano-gold particles within the nanoassembly can be stimulated once only a few of them are triggered by light. Thus, this design can overcome the shortcoming of low penetration of NIR, since not every nano-gold particle inside the nanoassembly needs to be hit by NIR.

Exemplary Definitions

In accordance with long standing patent law convention, the words “a” and “an” when used in this application, including the claims, denotes “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof that is pharmaceutically acceptable for administration to the relevant animal or acceptable for a therapeutic or diagnostic purpose, as applicable.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

The term “e.g.,” as used herein, is used merely by way of example, without limitation intended, and should not be construed as referring only those items explicitly enumerated in the specification.

The phrases “isolated” or “biologically pure” refer to material that is substantially, or essentially, free from components that normally accompany the material as it is found in its native state. Thus, isolated polynucleotides in accordance with the invention preferably do not contain materials normally associated with those polynucleotides in their natural, or in situ, environment.

As used herein, the term “kit” may be used to describe variations of the portable, self-contained enclosure that includes at least one set of reagents, components, or pharmaceutically-formulated compositions to conduct one or more of the diagnostic or therapeutic methods of the invention.

As used herein, the term “patient” (also interchangeably referred to as “host” or “subject”) refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a “patient” refers to any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, and any animal under the care of a veterinary practitioner.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids. These include the following: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to all amino acid chain lengths, including those of short peptides of from about 2 to about 20 amino acid residues in length, oligopeptides of from about 10 to about 100 amino acid residues in length, and polypeptides including about 100 amino acid residues or more in length. The term “sequence,” when referring to amino acids, relates to all or a portion of the linear N-terminal to C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide and polynucleotide chains, “sequence” and “subsequence” have similar meanings relating to the 5′-to-3′ order of nucleotides.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

### Multistage Vector-Mediated Thermal Ablation for the Treatment of Cancers

The results presented in the following example provide evidence for application of MSV delivery system in the treatment of TNBC by thermal ablation.

Materials and Methods

Fabrication of Discoidal pSi Particles and Surface Chemical Modification:

Discoidal pSi particles were fabricated as previously described with modifications (Ananta et al., 2010). The dried particles were then treated with Piranha Solution to obtain oxidized negatively charged silicon particles, and modified with 2% (vol./vol.) 3-aminopropyltriethoxysilane (APTES) (Sigma-Aldrich, St. Louis, Mo., USA) for 24 to 36 hrs at 55° C. to obtain positively-charged particles for loading of the nanoparticles. HAuNS synthesis has been reported previously (You et al., 2010; Lu et al., 2009). HAuNS was loaded into pSi by a combination of capillary force and surface charges.

Photothermal Effect in Aqueous Solution:

The GCSLX-05-1600m-1 fiber-coupled diode laser (DHC, China Daheng Group, Beijing, CHINA) with a center wavelength of 808±10 nm was powered by a DH 1715A-5 dual-regulated power supply. A 5-m×600-nm, core BioTex LCM-001 optical fiber (BioTex, Inc., Houston, Tex., USA) was used to transfer laser power from the laser unit to the target. The end of the optical fiber was attached to a retort stand using a movable clamp that was positioned directly above the sample cell. For measurement of temperature changes, NIR laser light was delivered through a quartz cuvette containing pSi, HAuNS, or pSi/HAuNS (100 mL). A thermocouple was inserted into the solution perpendicular to the path of the laser light. The temperature was measured over 10 min.

Photothermal Cytotoxicity In Vitro:

Cancer cells were seeded into a 96-well plate at a density of 10,000 cells/well. Free HAuNS or pSi/HAuNS were added into cell culture 5 hrs later after cells had adhered to the plate. Cells were then washed three times with serum-free medium the next day, and were irradiated with an NIR energy source at an output power of 2 W for 2 min (for SK-BR-3 cells), or 3 min (for MDA-MB-231 and 4T1 cells). Cells were washed three times with Hank's balanced salt solution 24 hrs after laser treatment, then stained using a Live/Dead Viability/Cytotoxicity kit from Invitrogen Corp./Life Technologies, Inc. (Carlsbad, Calif., USA) according to the manufacturer's protocol. Live cells were stained with calcein AM, while dead cells were stained with ethidium homodimer. Cells were then examined using an Olympus FluoView™ FV1000 confocal laser scanning microscope (FV1-ASW) equipped with filter sets specific for excitation/emission wavelengths at 494/517 nm for live cells (stained green) and 528/617 nm for dead cells (stained red).

Photothermal Therapy of Mammary Tumor in Nude Mice:

Six-week-old nude mice were inoculated with 4T 1 cells in the mammary gland fat pad. When tumors reached a size of 150-200 cm³, the mice were administered PBS, pSi, HAuNS, or pSi/HAuNS by intra-tumoral injection. Each mouse in the treatment group received 3×10⁸ pSi containing 1×10¹¹ HAuNS in 25 μL PBS. In the control groups, each mouse received 25 μL PBS, 3×10⁸ empty pSi, or 1×10¹¹ free HAuNS. The mice were treated with NIR energy (0.5 W for three min per tumor), and tumor growth was monitored for the next two weeks. All animals were sacrificed when tumor sizes in the PBS control group passed 1,000 cm³.

Monitoring Vascular Permeability:

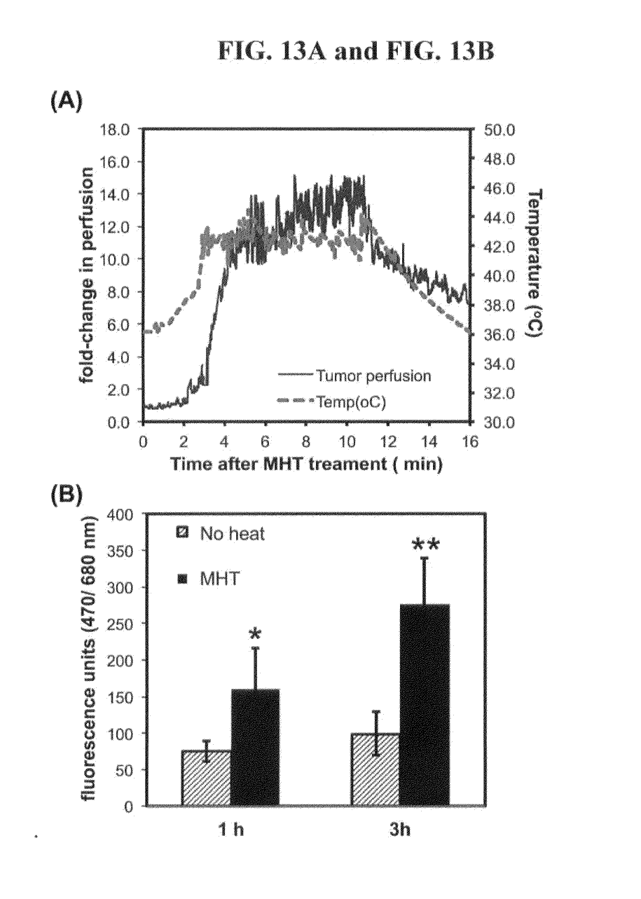

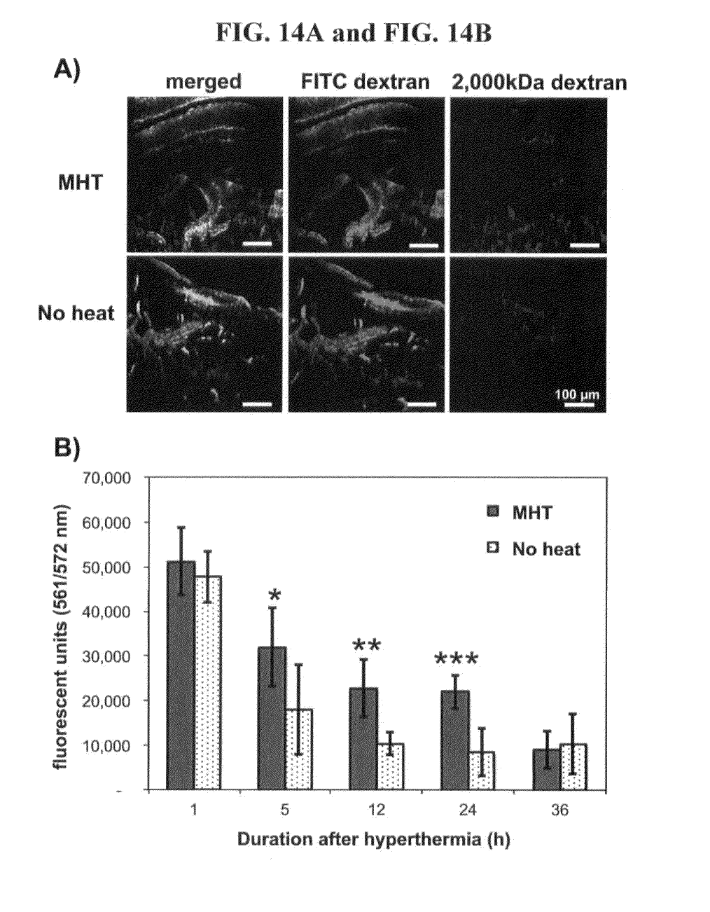

Tumor vascular function and changes in flow dynamics were evaluated by intravital microscopy (IVM) immediately after MHT treatment (within 5 min) or at specified end-points (FIG. 14A and FIG. 14B). Tumors were exposed using a skin-flap procedure described previously. Live animals were imaged on an upright Nikon A1R MP-ready laser scanning confocal microscope platform equipped with a resonance scanner, isoflurane anesthesia system, heated stage, and custom coverslip mounts. Anesthetized mice were administered with fluorescent dextran by retro-orbital injection (10 mg/kg, 50 μL PBS) and dye diffusion visualized by IVM for up to 60 min. The retro-orbital route was chosen over tail-vein injection due to its accessibility and reproducibility for quantitative imaging experiments. Fluorescent dyes were acquired with the following settings: FITC labeled dextran was excited at 488 nm while TRITC labeled dextran was excited at 561 nm collected at band-pass filters widths 30-50 nm centered at 525 nm for FITC- and 579 nm for TRITC-labeled dextran. 512×256-bit, two-channel images of s.c. tumors were acquired over multiple field of views (FOVs) from the time of dye injection using a 4× objective lens with a pinhole of 1.0 Airy units. To minimize local phototoxicity, and to compensate for tissue heterogeneity, non-overlapping FOVs were randomly selected and imaged at a 20-sec interval to reduce photobleaching. The camera and acquisition settings were kept constant between different treatment groups. Animals were sacrificed, tumor excised and prepared for histological and spectrofluorometric analyses to confirm the effect of hyperthermia on dye extravasation (FIG. 13A and FIG. 13B).

Results and Discussion

A pSi nanoassembly was developed with hollow gold nanoshells (HAuNS) to explore photothermal effects for therapeutics taking advantage of the nanoscale effect. HAuNS are favorable over the solid gold nanoparticle (AuNP), since the near infrared (NIR) light for hollow gold has a deep penetration inside the body and causes less damage to tissues due to less absorbance by the tissue chromophores. Human and mouse breast cancer lines were used to test cell killing in vitro and the mouse model of 4T1 mammary tumor for in vivo studies.

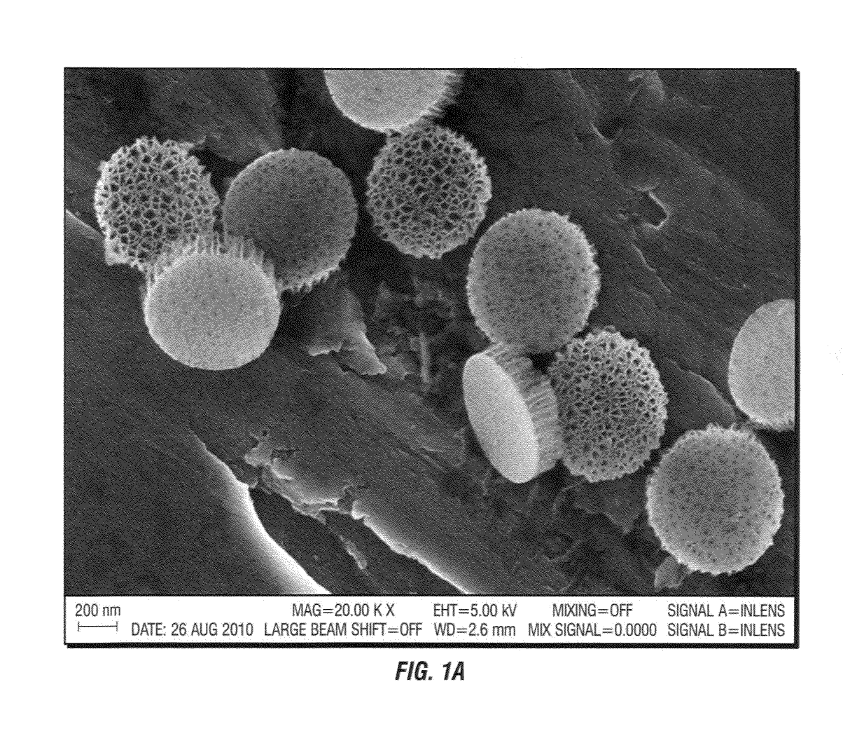

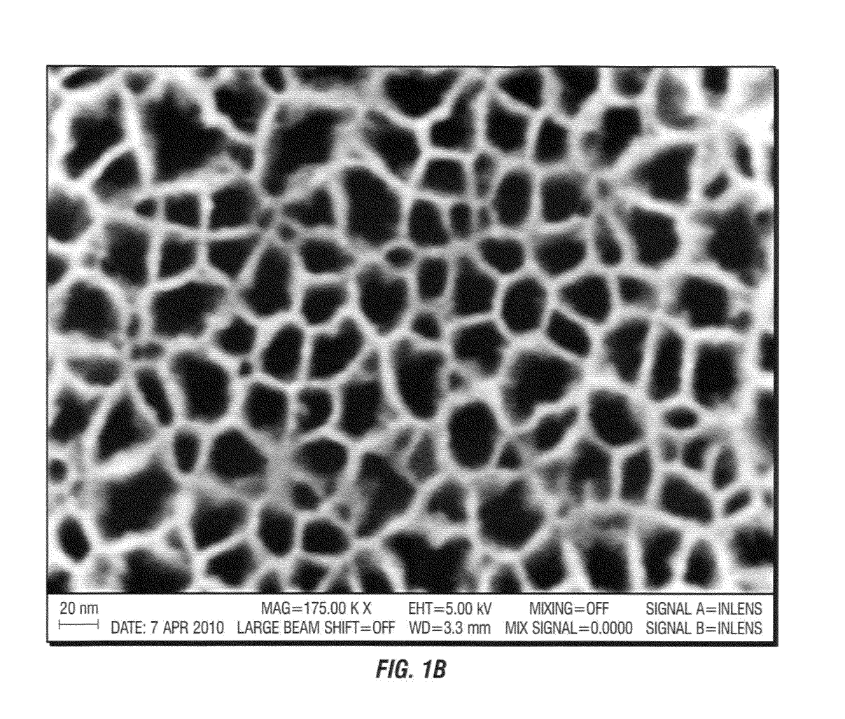

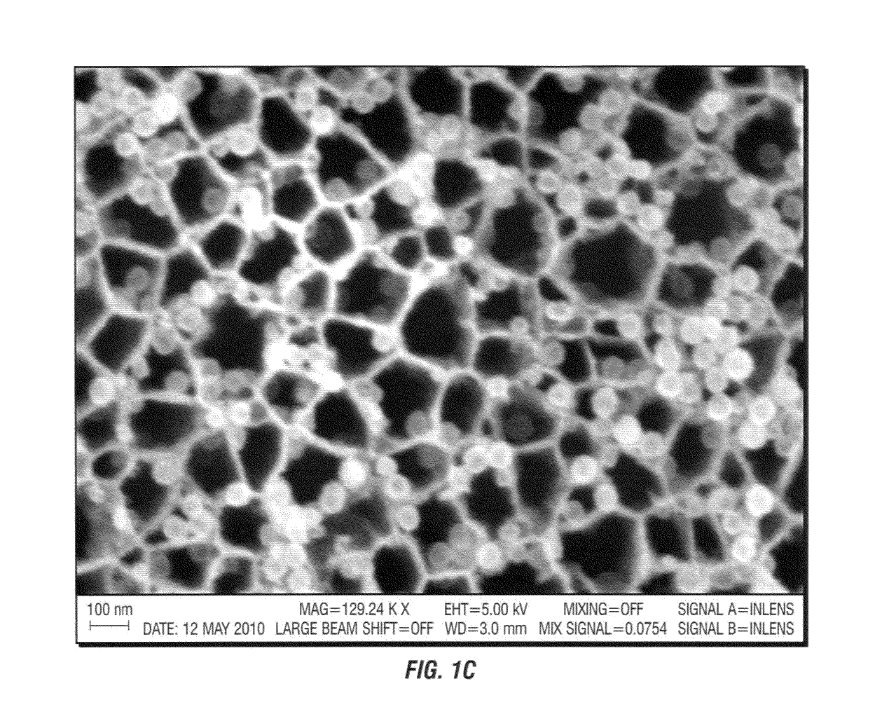

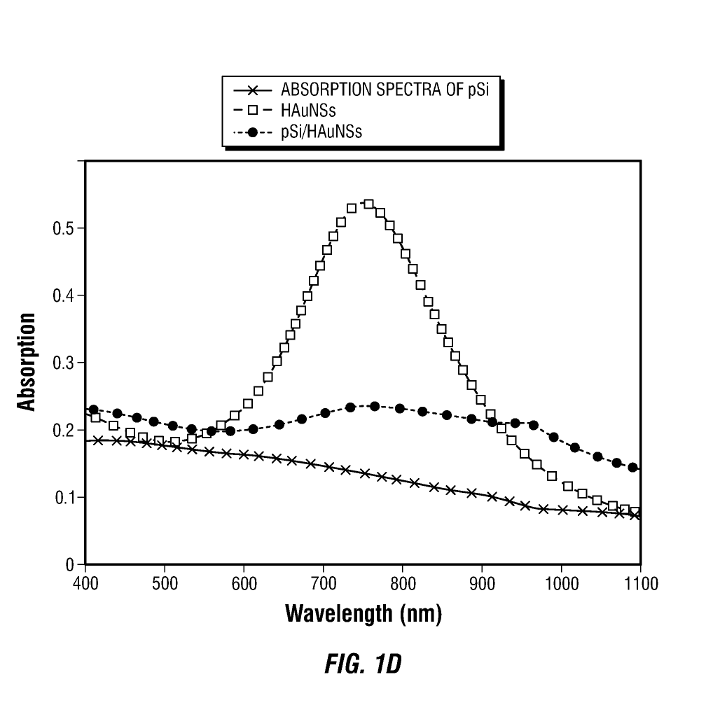

pSi particles were fabricated over three major steps: formation of porous silicon films, photolithographic patterning of particles and reactive ion etch. The porous structure of particles was tailored by electrochemical etching to a mean pore size of 60 nm and a porosity of about 80%, while the particle sizes were precisely defined by photolithography to 1000 nm in diameter and 400 nm in thickness. SEM images revealed that the pores were evenly distributed across the whole area (FIG. 1A and FIG. 1B). Since the surface of the pSi particles were conjugated with APTES, they were positively charged, which facilitated loading of negatively-charged HAuNS through favorable electrostatic interactions (FIG. 1C). Multiple HAuNS particles could be observed in each pore across the entirety of the silicon microparticle.

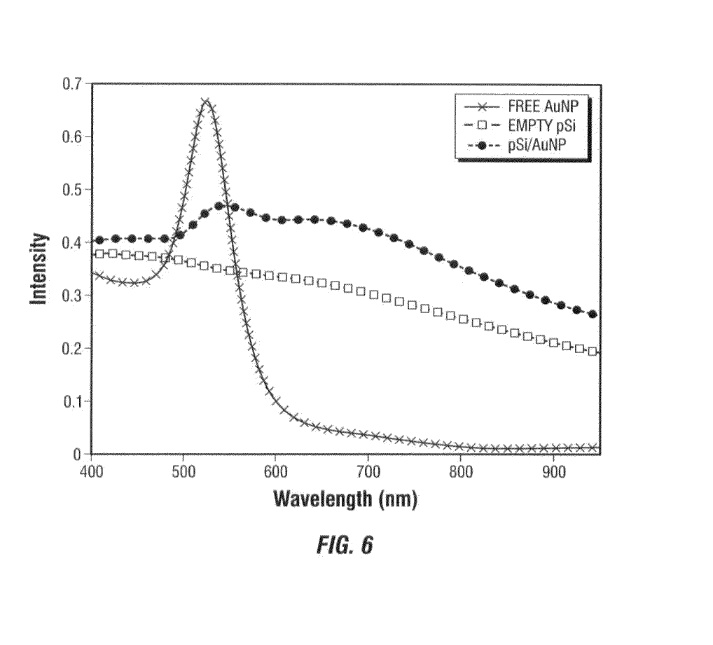

HAuNS particles of 28-nm average diameter showed a plasma resonance peak at 750 nm (FIG. 1D), which is similar to that observed for most HAuNS particles of similar size (You et al., 2010 and Lu et al., 2009). This peak disappeared when the HAuNS particles were loaded into pSi particles. There was a small peak around 950 nm indicating a red-shift of absorbance from pSi/HAuNS (FIG. 1D), while empty pSi particles did not have any significant absorption in the 400-1100 nm range. Absorption spectra of solid gold nanoparticles (AuNP) were measured with a plasma resonance peak at 528 nm (FIG. 6). Loading of AuNP into pSi also resulted in disappearance of the peak and a red shift of the small peak in the 600-750 nm range.

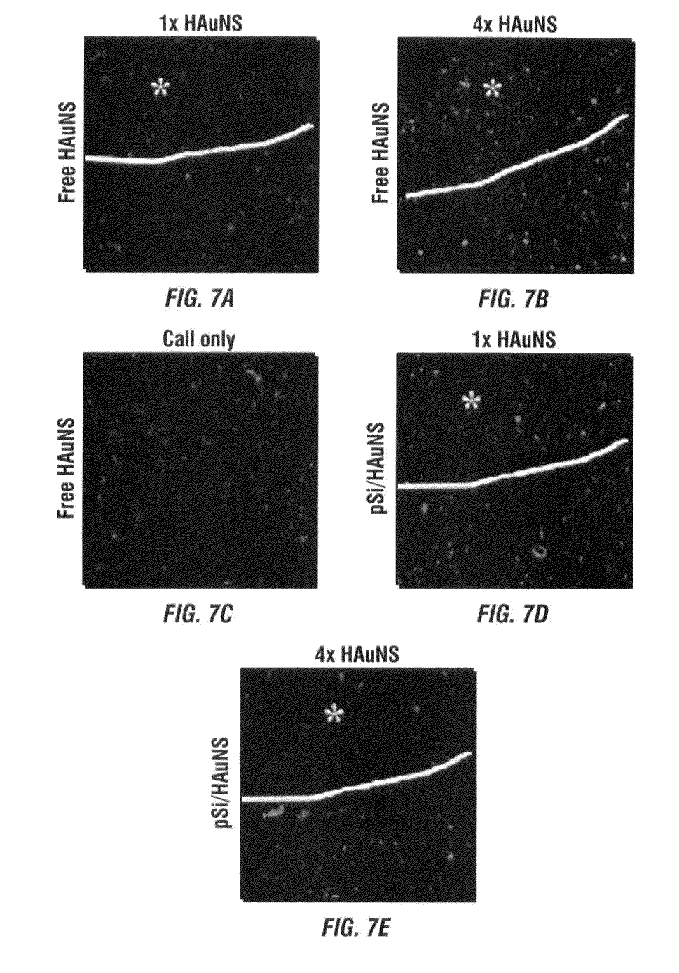

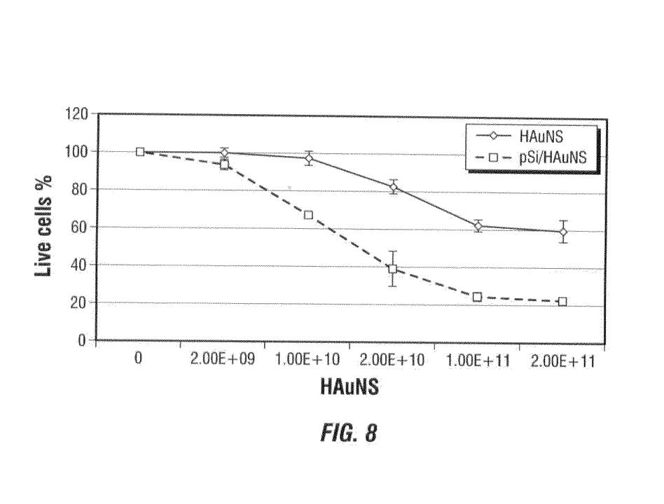

In a study with SK-BR-3 (FIG. 8), cells were seeded into a 96-well plate at a density of 10,000 cells/well; free HAuNS or pSi/HAuNS was added to the cell culture 5 hrs later, after cells had adhered to the plate. Cells were then washed three times with serum-free medium the next day, and were irradiated with NIR energy at an output power of 2 W for 2 min. Live cells were measured with the MTT reagent 24 hrs later. A simple mix of AuNP with silicon did not result in disappearance of the plasma resonance peak (FIG. 7A-FIG. 7E). The most plausible explanation for vanishing absorption from the pSi/HAuNS nanoassembly is a scattering effect from the pSi microparticle.

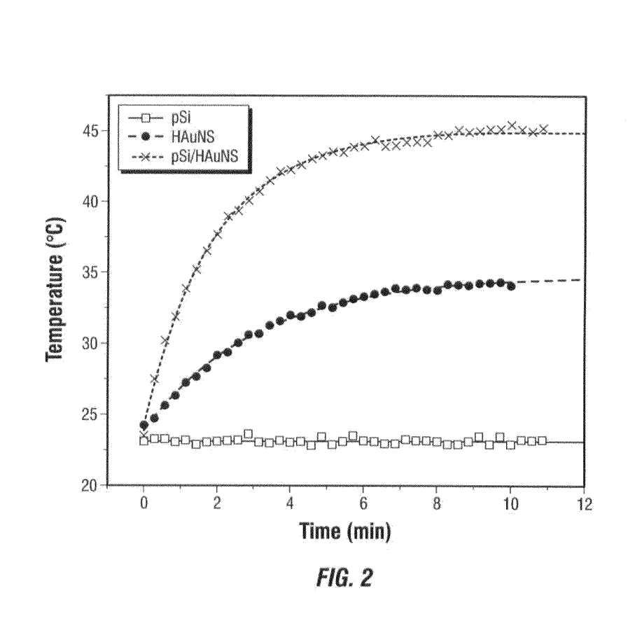

Water suspension of the particles was used to measure heat generation triggered by a NIR laser. pSi particle alone did not show any heat generation as expected and stayed at room temperature all the time with the NIR laser continuously on (FIG. 2). The temperature in the HAuNS colloidal suspension increased by 10.9° C. and reached a steady level of 34.7° C. within 10 min. A larger increase in temperature was observed in the pSi/HAuNS suspension. The temperature reached 45° C. within seven min—almost twice as high as that obtained when using equivalent amounts of colloidal HAuNS. There was an overall increase of 20.6° C. above room temperature. Time constants for heat generation kinetics were calculated at 3.1 s for HAuNS and 1.9 s for pSi/HAuNS.

To test whether the enhanced thermal generation could be translated into efficient cell killing, cancer cells were treated with free gold nanoparticles or pSi/HAuNS, and monitored cell growth by the MTT assay. pSi and AuNP were used as controls. AuNP particles were not expected to have any effect on thermal cytotoxicity, as the NIR laser used in the study (wavelength of 808 nm) did not have any effect on solid gold particles.

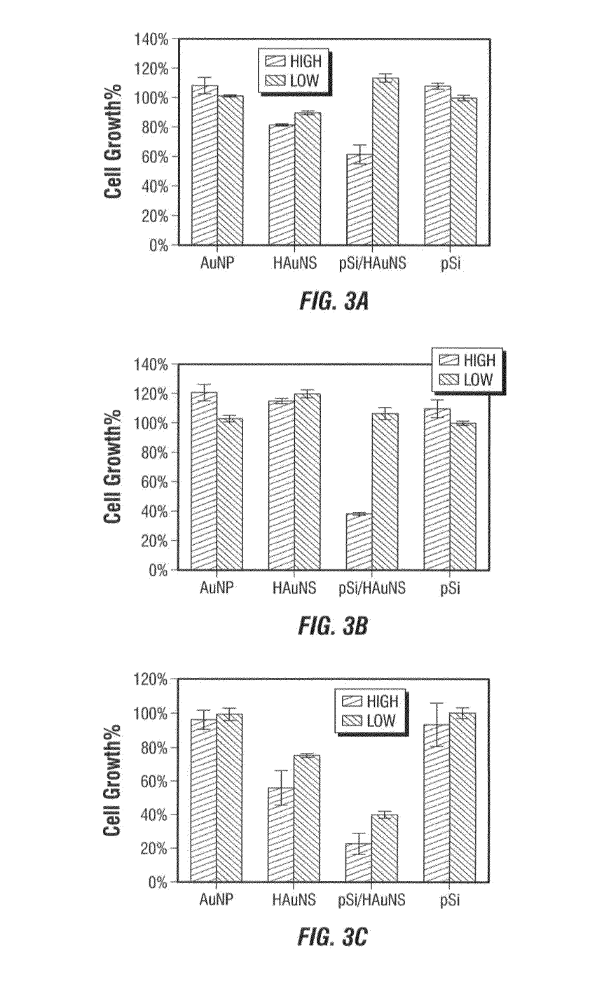

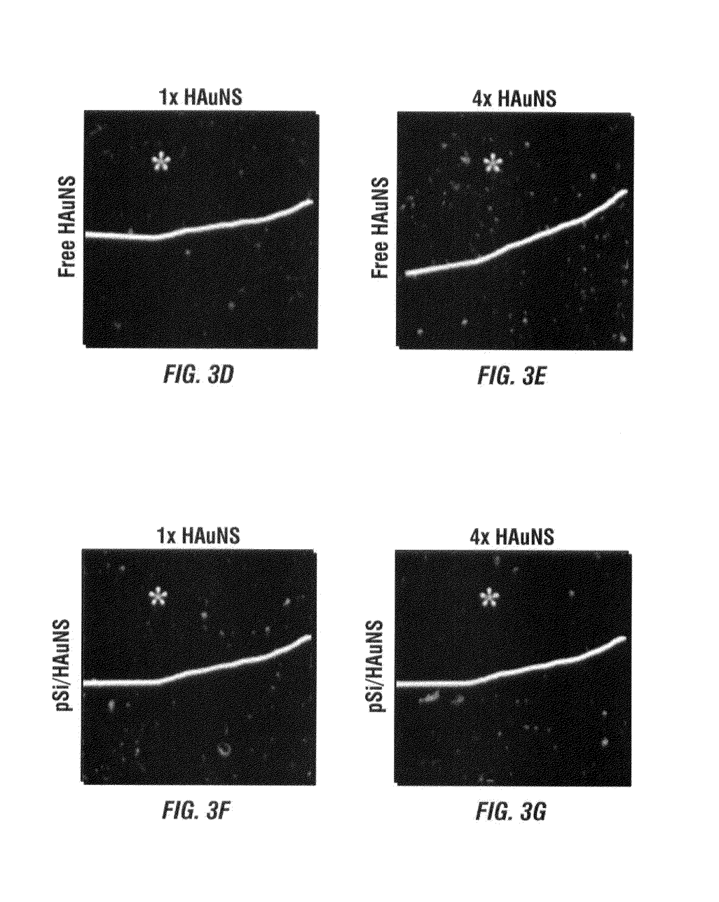

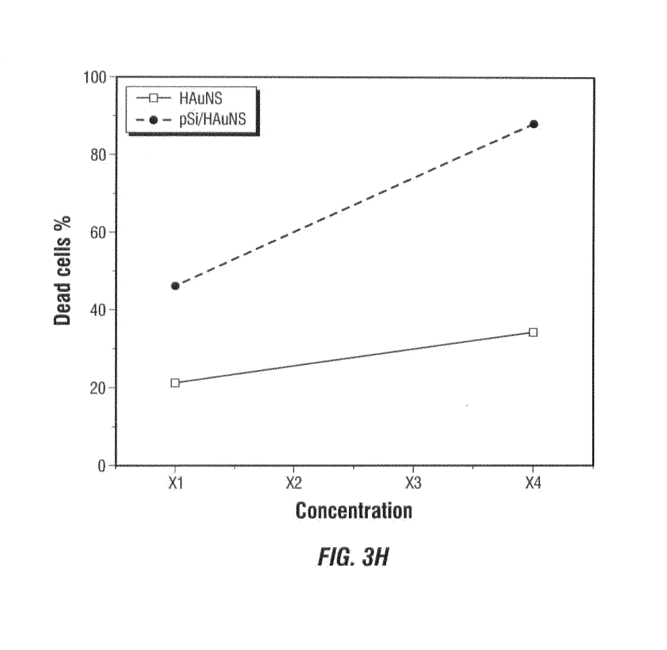

Different amount of HAuNS were loaded into a fixed number of silicon particles (2×10⁹ HAuNS or 2×10¹⁰ HAuNS in 1×10⁸ pSi), so that any changes in cell growth would be due to the effects of HAuNS, and not pSi particles themselves. As expected, neither free HAuNS nor pSi/HAuNS had any significant impact on cell growth when there was not enough HAuNS present for sufficient heat generation (FIG. 3A-FIG. 3C). However, when the number of gold particles increased, pSi/HAuNS particles were very effective in killing the cancer cells, although the effects obtained using free HAuNS was less significant (FIG. 3A-FIG. 3C). A further increase in the number of HAuNS particles resulted in cell killing—both using free gold and with the gold-silicon nanoassembly. A similar trend was observed using the MDA-MB-231 and SK-BR-3 human breast cancer cells (FIG. 3A and FIG. 3B) and the 4T1 murine mammary tumor cells as a target (FIG. 3C). These results indicated that the thermal ablation effect was generalized, since the tested cell lines carried significant genetic backgrounds, and had diverse mutation spectra. For example, the SK-BR-3 cells overexpress the HER2 gene, while MDA-MB-231 is a triple-negative cell line that lacks expression of the estrogen receptor, the progesterone receptor, and the HER2 gene product.

Two dyes were used to detect cell viability after exposure to NIR laser. The nonfluorescent calcein AM was converted to the intensely green fluorescent calcein in live cells. Ethidum homodimer-1 (EthD-1) entered dead/dying cells through damaged membranes and bound to nucleic acid, producing a bright red fluorescence. Cells were treated with a low dose (1×, 6.25×10⁹ HAuNS/well) and a high dose (4×, 2.5×10¹⁰ HAuNS/well) free HAuNS or pSi/HAuNS. Around 20% cells were positive for EthD-1 staining without NIR laser treatment. The free HAuNS-treated cells did not undergo significant cell death at the 1× dose (FIG. 3D). The amount of EthD-1-positive cells doubled with the 4× HAuNS dose (FIG. 3E); however, the percentage of dead cells was not as high as in the sample treated with the 1× dose pSi/HAuNS (FIG. 3D vs. FIG. 3E). Up to 88% cancer cells treated with 4×pSi/HAuNS were EthD-1-positive 24 hours after NIR treatment (FIG. 3F), comparing to less than 35% EthD-1-positive cells treated with 4× HAuNS (FIG. 3G). The remaining cells would mostly undergo apoptosis in the following days. Quantitative analysis of cell death from these treatments is shown in FIG. 3H. This result was in agreement with the thermal generation result that showed the increase of temperature was three times as fast from the same amount of HAuNS in nanoassembly as from free HAuNS. These results indicated that enhanced cancer cell killing could be achieved by packaging gold nanoshells inside pSi particles.

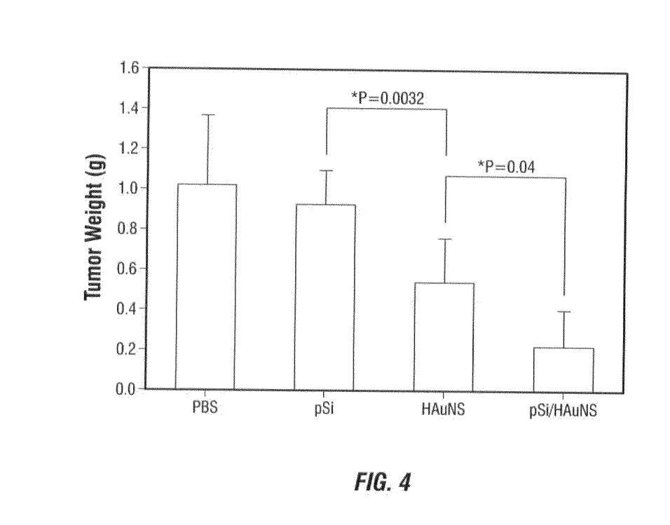

In an important study, murine 4T1 tumor mice were generated to test the efficacy of thermal ablation by pSi/HAuNS on tumor growth. When tumor sizes reached 150-200 cm³, free HAuNS or pSi/HAuNS were delivered by intra-tumoral injection. The tumors were treated with NIR laser the next day, and tumor growth was monitor in the next 10 days before the mice were sacrificed. A single treatment of thermal ablation from both free HAuNS and pSi/HAuNS significantly inhibited tumor growth (FIG. 4). However, the pSi/HAuNS-treated mice did not have much tumor growth, while the tumor weight in the free HAuNS group more than doubled during the same period. In the pSi control group, the average tumor weight almost quadrupled to 1 g/tumor. This result clearly supported the use of the pSi/HAuNS nanoassemblies as primary cancer therapeutics.

Previous studied have demonstrated the potential application of pSi as a multistage vector for the delivery of therapeutics and/or imaging agents (Tanaka et al., 2010; Ferrari, 2010; Ananta et al., 2010). Due to the nature of the size and porosity, pSi has been demonstrated to be useful as a vehicle for delivering numerous therapeutic agents, and can achieve tumoritropic accumulation independent of the EPR effect. In this example, pSi was used not only for enhanced tumor localization, but also as an essential part of the therapeutic complex for the enhancement of thermal ablation, making it a part of therapeutic mechanism. The kinetics of heat generation by HAuNS was analyzed with respect to heat exchange. Theoretical models of gold nanoparticle suspensions have been developed (Richardson et al., 2009; Roper et al., 2007), where the energy balance was governed by laser induced heat through electron-phonon relaxations on HAuNS surfaces and heat dissipation.

The temperature profile generated by laser was derived by introducing the rate of energy adsorption, A [K/s], and the rate of heat loss, B [1/s] Richardson et al. (2009):

$$T ( t ) = {T}_{0} + \frac{A}{B} ( 1 - exp ( - Bt ) ) \qquad Eq . ( 1 )$$

Equation (1) has been fitted to temperature profiles in FIG. 2, where A=4.17 K/s and B=0.36 s⁻¹ for HAuNS and A=12.26 K/s and B=0.56 s⁻¹ for pSi/HAuNS. The rate of heat generation was 3 times as high for pSi/HAuNS as that of free HAuNS. It was anticipated that both HAuNS and pSi/HAuNS would have similar heat loss rates, since the only difference was the inclusion of pSi in the nanoassembly. Interestingly, the heat dissipation rate was also higher for the pSi/HAuNS nanoassembly. Thus, the model in Equation (1) that was validated for colloidal gold could not explain the pSi/HAuNS temperature increase curve using the same heat loss rates. This suggests that thermal equilibrium of pSi/HAuNS is defined by different thermal properties of the system.

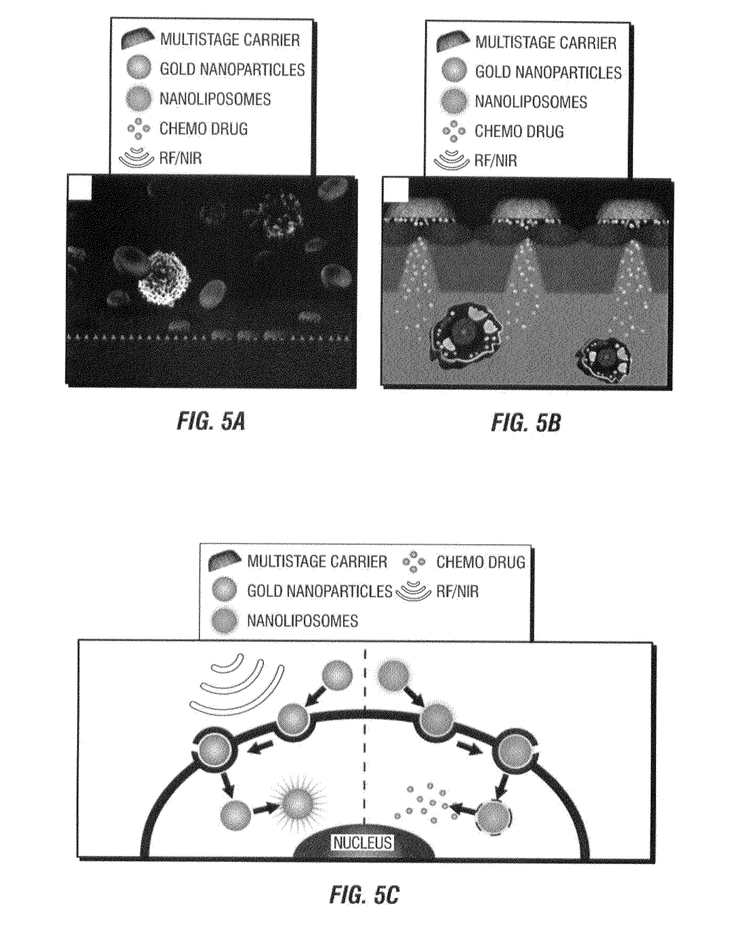

The clear difference between HAuNS and pSi/HAuNS was the distribution of the gold nanoparticles, which is summarized in FIG. 5A-FIG. 5C. Based on the amount of HAuNS and pSi used in this study, it was concluded that the average gold inter-particle distance in colloid HAuNS was 1.7 μm, and free pSi particles were separated by 11 μm. In the pSi/HAuNS nanoassembly, however, HAuNS were fixed within pSi with a domain of less than 1 μm (FIG. 1). It has been reported that surface plasmons penetrate dielectric media up to hundreds of nanometers away from the metal surface (Huang et al., 2010; Jensen et al., 1999), and that specific orientation of HAuNS can transfer energy over hundreds of nanometers (Maier et al., 2003). Thus, HAuNS particles within the nanoassembly should become electromagnetically coupled mostly through dipole-dipole interactions (Rechberger et al., 2003), and can function as waveguide-like structures that increase energy transfer and heat production as illustrated in FIG. 5A-FIG. 5C. To support this finding, increased heat production in HAuNS structures through varying the angle of incident photon beam has already been observed (Govorov et al., 2006). Furthermore, bringing HAuNS closer to clusters can cause a red shift of surface polaron resonance (Hovel et al., 1993), which could be over 100 nm (Jensen et al., 1999). The peak around 950 nm found in pSi/HAuNS, which was not observed in free HAuNS suspension, indicates a red shift of 200 nm. The increased wavelength in the NIR region can therefore be used for deeper penetration into tissues, where HAuNS are arranged in a collective nanoscale structure. Potentially, the enhanced heating effects could also be supplemented by scattering that increases light path.

Since convective flows inside pSi are negligible, thermal diffusion is the most important aspect. Characteristic thermal diffusion lengths of water, expressed as over given time t and the thermal diffusivity D=1.4×10⁻³ cm²/s, are 0.7 μm over 1 μs and 23.4 μm over 1 ms. Therefore, using sub-microsecond range NIR pulses, thermal expansion domains around individual HAuNS will start to overlap, while NIR pulses over 1 μs will make pSi/HAuNS a continuous, thermally-excited domain (thermal spot-source). Silicon has almost six times as high thermal diffusivity as water. At 80% porosity in pSi, the effective thermal diffusivity is 3.3×10⁻³ cm²/s. It has already been shown that thermal dissipation of gold nanoparticles can be increased by wrapping gold core with silica shell (Hu et al., 2003). Therefore, L_{T} of pSi increases by almost 50% that makes heat dissipation even more efficient within premises of pSi, suggesting that induced photothermal effects could be enhanced by thermal properties of the HAuNS organizing materials.

These results have demonstrated the superior photothermal ablation effect from pSi/HAuNS. The inventors have shown that pSi/HAuNS nanoassemblies were much more efficient at producing localized heat generation in response to NIR excitation than free gold nanoparticles alone. Moreover, the pSi/HAuNS nanoassembly compositions described herein offer the unexpected benefits of red-shift, which permits access to deeper tissues and also leads to a more efficient energy-to-heat conversion. These beneficial properties have been achieved by exploiting nanoscale organization features of HAuNS, and demonstrate for the first time therapeutic applications of HAuNS compositions, and particularly their facility in thermal ablation and other forms of localized thermotherapy.

### Example 2

### Assembly of MSVs-Nanorods

Multistage vehicles (MSVs) are porous particles that allow the packaging and the delivery of therapeutics to targeted sites. Coating MSVs with elements with strong plasmonic properties (such as gold or silver) result in particles that can absorb and convert energy efficiently. Assembly of gold nanostructures such as gold nanorods (Au-NRs) on the surface of MSVs imparts strong plasmonic properties. By harnessing this novel property, the MSVs can serve not only as drug delivery vehicles, but also as thermal depots that can be utilized for remotely triggered therapeutic release. Other applications include, without limitation, combinational therapies, in which both the therapeutic agent is delivered and heat is generated to synergize cancer therapy.

Materials and Methods

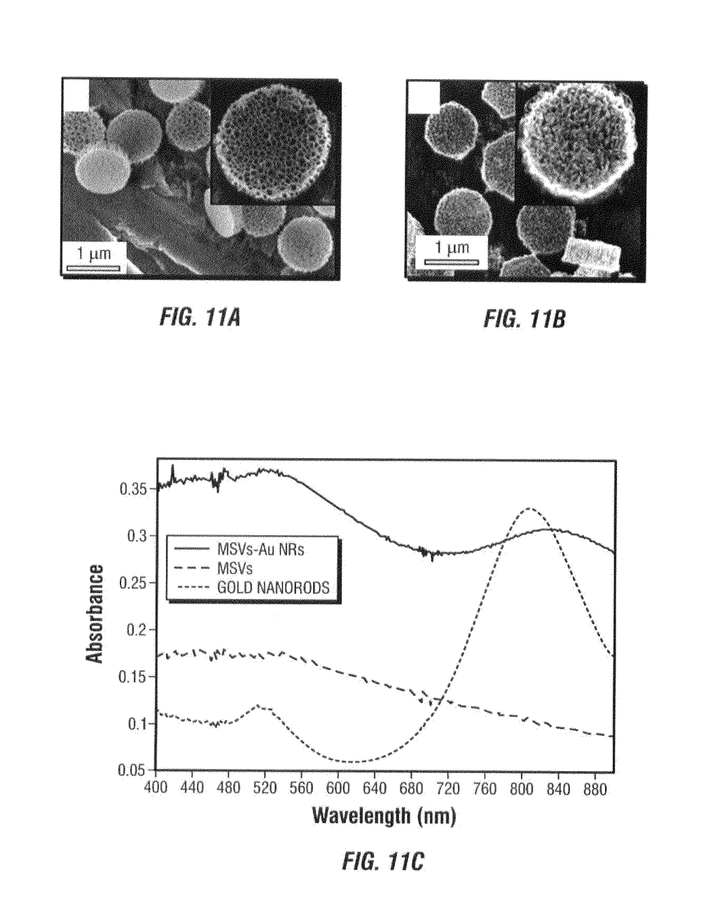

The assembly of MSVs-Au-NRs particles is a two-step process in which MSVs with a number of different functionalities (i.e., hydroxyl or amine groups) can be prepared. The surface modification determines the coating strategy employed to immobilize gold onto its surface. For instance, negatively charged MSVs (i.e., carboxyl) were electrostatically coated with positively-charged, aminated gold particles to form stable, uniform gold-MSVs that serve dual functions—both as delivery vehicles and as thermal ablative agents) (see e.g., FIG. 11A, FIG. 11B, and FIG. 11C).

Formation of MSVs-Gold Nanorod Hybrid Particles

The following protocol was used to coat positively-charged MSVs with negatively-charged gold nanorods.
100 μL of APTES-modified MSVs (1×10⁹/mL) suspended in isopropyl alcohol (IPA) was centrifuged and then resuspended in 100 μL of saline buffer.
20 μL of gold nanorods (1×10¹²/mL in PBS) was sonicated for 5 min and then added to APTES-modified MSVs.
The composition was then gently mixed for 1 hr, after which the gold-coated MSVs were recovered by centrifugation (2,000 rpm, 5 min) A pellet of MSVs-Au-NRs was collected and re-suspended in 200 μL, then re-centrifuged 3 times.
Purified MSVs-Au-NRs particles were then centrifuged (2,000 rpm, 10 min) and maintained at 4° C. for long-term storage.

While the protocol detailed above is used to couple positively charged MSVs to negatively charged gold NRs, it can be extended to assemble particles of opposite charges (i.e., negatively charged MSVs and positively charged gold nanorods). The assembly of hybrid particles using this strategy is primarily by electrostatic interactions. The formed bonds are weak, and detach faster ensuring a more rapid release of payload from the pores of the MSVs. Such formulations are best suited for applications where faster, triggered drug release is desired.

Covalent Ligation of MSVs to Gold Nanorods

Assembly of MSVs and gold nanorods with stronger bonds that are formed through covalent ligation are, also, however possible in accordance with particular aspects of the present invention. For example, strongly linked MSVs-gold nanorod structures may be suitable for clinical applications where a prolonged and/or sustained (and optionally, a heat-assisted) release may be desired. For instance, a facile heterofunctional linker such as Traut's agent (Pierce Biochemical Co., MA, USA) can be used to ligate hydroxyl-terminated MSVs to bare gold nanorods. This reagent reacts with the hydroxyl on the surface of MSVs, and forms gold-thiol bonds with bare gold.

The following protocol was used to coat hydroxyl terminated MSVs are reacted with bare gold nanorods to form stable and hybrid particles
100 μL of hydroxyl-modified MSVs (1×10⁹/mL) suspended in isopropyl alcohol (IPA) was centrifuged and then resuspended in 100 μL of saline buffer.
30 μL of bare gold nanorods (1.5×10¹²/mL in PBS) was sonicated for 5 min and then added to a solution of hydroxyl-terminated MSVs.
Mixture was sonicated for 5 min and then added into a freshly prepared solution of Traut's reagent (2 mM, pH=8.5).
The reaction was aided by intermittent sonication for 1 hr, after which gold-coated MSVs were recovered by centrifugation (2,000 rpm, 5 min). A pellet of MSVs-Au-NRs was collected, and re-suspended in 200 μL and re-centrifuged 3 times.
Purified MSVs-NRs particles were recovered by centrifugation (2,000 rpm, 10 min), then maintained at 4° C. for long-term storage.

Results and Discussion

The schematic representation of covalent ligation of MSVs to gold nanorods is shown in FIG. 10. This assembly is a one-step reaction process where the linker is added to mixture at pH=8.5 and incubated for a short duration.

The assembly of MSVs-gold nanorods by electrostatic interaction is illustrated in FIG. 11A, FIG. 11B, and FIG. 11C. As is observed in FIG. 11B, the MSVs are densely coated with gold nanorods which are stable even after several rounds of centrifugation. Spectral properties of the resulting hybrid particles are characterized in FIG. 11C. The spectrum shows an enhancement of near-infrared absorption (800-900 nm) when compared to the absorbance of MSVs alone. This indicates that the hybrid particles can efficiently absorb near infrared energy. This creates an attractive platform with potential uses for heat-assisted therapy as well as for drug delivery applications.

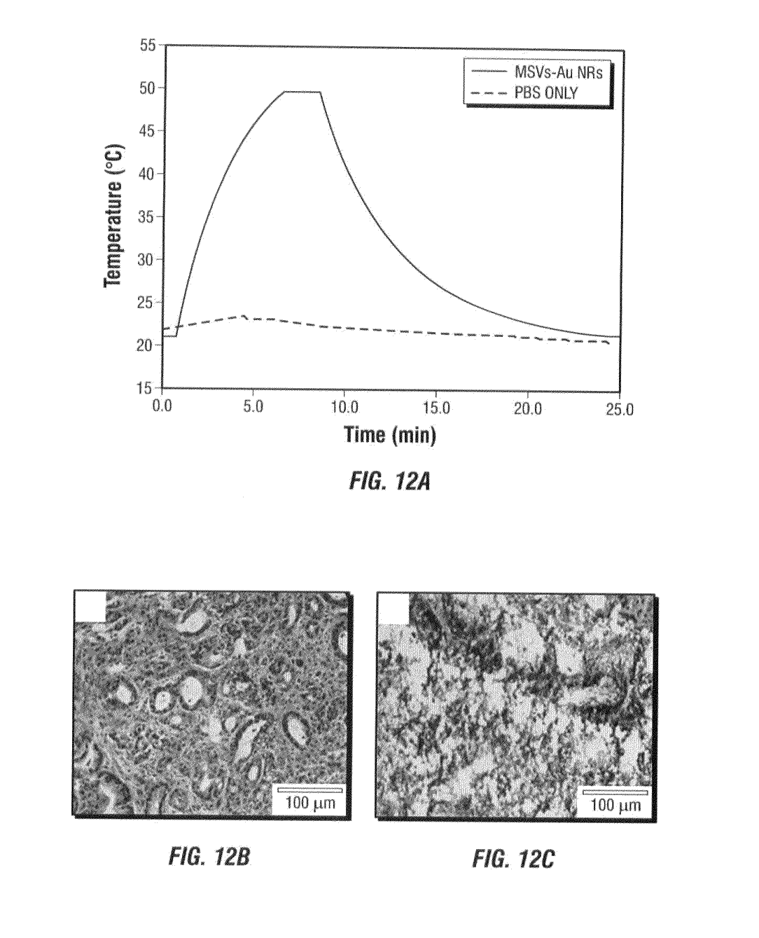

The ability of the ‘as-prepared’ MSVs-NRs hybrid particles to convert energy and heat up is illustrated in FIG. 12A. In this study, a diluted solution of MSVs-NRs (1×10⁸ MSVs, 100 μL saline solution) was exposed to 810-nm laser irradiation, and a rapid increase in temperature was observed. A high-temperature differential (+25° C.) relative to the heating of an equal volume of saline solution was seen. By forming these hybrid particles, an additional novel property has been imparted to the function of MSVs making them useful for both drug delivery applications as well as for thermal therapy or for heat-assisted controlled drug activation or release applications. The use of hybrid particles to induce selective thermal damage in cancer cells is shown in FIG. 12B and FIG. 12C.

### REFERENCES

The following references, to the extent that they provide exemplary procedural or other details supplementary to those set forth herein, are specifically incorporated herein in their entirety by express reference thereto:
United States Patent Application Number 2010/0074958.
United States Patent Application Number 2010/0029785.
United States Patent Application Number 2008/031182.
United States Patent Application Number 2008/0280140.
United States Patent Application Number 2008/0206344.
United States Patent Application Number 2008/0102030.
United States Patent Application Number 2003/0114366.
Alivisatos, A P, “Semiconductor clusters, nanocrystals, and quantum dots,” Science, 271(5251):933-937 (1996).
Ananta, J S et al., “Geometrical confinement of gadolinium-based contrast agents in nanoporous particles enhances T1 contrast,” Nat. Nanotechnol., 5(11):815-821 (2010).
Atkinson, R L, et al., “Thermal enhancement with optically activated gold nanoshells sensitizes breast cancer stem cells to radiation therapy,” Sci. Translat. Med., 2(55):55ra79 (2010).
Balak, M N et al., “Novel D761Y and common secondary T790M mutations in epidermal growth factor receptor—mutant lung adenocarcinomas with acquired resistance to kinase inhibitors,” Clin. Cancer Res., 12:6494-6501 (2006).
Bean, J et al., “MET amplification occurs with or without T790M mutations in EGFR mutant lung tumors with acquired resistance to gefitinib or erlotinib,” Proc. Natl. Acad. Sci. USA, 104:20932-7 (2007).
Bloch, S H et al., “Optical observation of lipid- and polymer-shelled ultrasound microbubble contrast agents,” Appl. Phys. Lett., 84(4):631-633 (2004).
Cohen, M H et al., “Microfabrication of silicon-based nanoporous particulates for medical applications,” Biomed. Microdevices, 5(3):253-259 (2003).
Decuzzi, P et al., “Size and shape effects in the biodistribution of intravascularly injected particles,” J. Control. Release, 141(3):320-327 (2010).
Decuzzi, P, and Ferrari, M, “Design maps for nanoparticles targeting the diseased microvasculature,” Biomaterials, 29(3):377-384 (2008).
Dickerson, E B et al., “Gold nanorod assisted near-infrared plasmonic photothermal therapy (PPTT) of squamous cell carcinoma in mice,” Cancer Lett., 269(1):57-66 (2008).
Duncan, R, “The dawning era of polymer therapeutics,” Nat. Rev. Drug Discov., 2:347-360 (2003).
Eberhard, D A et al., “Mutations in the epidermal growth factor receptor and in KRAS are predictive and prognostic indicators in patients with non-small-cell lung cancer treated with chemotherapy alone and in combination with erlotinib,” J. Clin. Oncol., 23(25):5900-5909 (2005).
Engelman, J A et al., “MET amplification leads to gefitinib resistance in lung cancer by activating ERBB3 signaling,” Science, 316:1039-43 (2007).
Ferrari, M, “Cancer nanotechnology: opportunities and challenges,” Nat. Rev. Cancer, 5(3):161-171 (2005).
Ferrari, M, “Experimental therapies: vectoring siRNA therapeutics into the clinic,” Nat. Rev. Clin. Oncol., 7(9):485-6 (2010).
Ferrari, M, “Frontiers in cancer nanomedicine: directing mass transport through biological barriers,” Trends Biotechnol., 28:181-8 (2010).
Glazer, E S and Curley, S A, “Radiofrequency field-induced thermal cytotoxicity in cancer cells treated with fluorescent nanoparticles,” Cancer, 116(13):3285-3293 (2010).
Goldsmith, H S and Turitto, V T, “Rheological aspects of thrombosis and haemostasis: basic principles and applications,” Thromb. Haemost., 55:415-435 (1986).
Govorov, A. O. et al., “Gold nanoparticle ensembles as heaters and actuators: melting and collective plasmon resonances,” Nanoscale Res. Lett., 1:84-90 (2006).
Gradishar, W. J. et al., “Phase III trial of nanoparticle albumin-bound paclitaxel compared with polyethylated castor oil-based paclitaxel in women with breast cancer,” J. Clin. Oncol., 23:7794-7803 (2005).
Hirsch, L. R. et al., “Nanoshell-mediated near-infrared thermal therapy of tumors under magnetic resonance guidance,” Proc. Natl. Acad. Sci. USA, 100(23):13549-13554 (2003).
Hövel, H et al., “Width of cluster plasmon resonances: bulk dielectric functions and chemical interface damping,” Phys. Rev. B., 48(24):18178-18188 (1993).
Hu, M et al., “Heat dissipation in gold-silica core-shell nanoparticles,” Chem. Phys. Lett., 372:767-772 (2003).
Huang, C J et al., “Long range surface plasmon and hydrogel optical waveguide field-enhanced fluorescence biosensor with 3D hydrogel binding matrix: On the role of diffusion mass transfer,” Biosens. Bioelectron., 24(4):1425-1431 (2010).
Ibrahim, N K et al., “Multicenter phase II trial of ABI-007, an albumin-bound paclitaxel, in women with metastatic breast cancer,” J. Clin. Oncol., 23(25):6019-6026 (2005). Jemal, A et al., “Cancer statistics, 2010,” CA Cancer J. Clin., 60(5):277-300 (2010).
Jensen, T et al., “Electrodynamics of Nobel metal nanoparticles and nanoparticle clusters,” J. Cluster Sci., 10(2):295-297 (1999).
Jones, S et al., “Core signaling pathways in human pancreatic cancers revealed by global genomic analyses,” Science, 321(5897):1801-1806 (2008).
Kennedy, L C et al., “A new era for cancer treatment: gold-nanoparticle-mediated thermal therapies,” Small, 7(2):169-183 (2011).
Kim, B. et al., “Tuning payload delivery in tumour cylindroids using gold nanoparticles,”Nat. Nanotechnol., 5(6):465-472 (2010).
Kirui D K et al., “Tumor vascular permeabilization using localized mild hyperthermia to improve macromolecule transport,” Nanomedicine, PMID: 24262998), In Press (2014).
Kobayashi, S et al., “EGFR Mutation and resistance of non-small-cell lung cancer to gefitinib,” N Engl. J. Med., 352:786-792 (2005).
Lu, W et al., “Targeted photothermal ablation of murine melanomas with melanocyte-stimulating hormone analog-conjugated hollow gold nanospheres,” Clin. Cancer Res., 15(3):876-886 (2009).
Lynch, T J et al., “Activating mutations in the epidermal growth factor receptor underlying responsiveness of non-small-cell lung cancer to gefitinib,” N Engl. J. Med., 350(21):2129-2139 (2004).
Maeda, H, “The enhanced permeability and retention (EPR) effect in tumor vasculature: the key role of tumor-selective macromolecular drug targeting,” Adv. Enzyme Regul., 41:189-207 (2001).
Maier, S A et al., “Local detection of electromagnetic energy transport below the diffraction limit in metal nanoparticle plasmon waveguides,” Nat. Mater., 2:229-232 (2003).
O'Neal, D P et al., “Photo-thermal tumor ablation in mice using near infrared-absorbing nanoparticles,” Cancer Lett., 209:171-176 (2004).
Oyewumi, M O et al., “Comparison of cell uptake, biodistribution, and tumor retention of folate-coated and PEG-coated gadolinium nanoparticles in tumor-bearing mice,” J. Control. Release, 95(3):613-626 (2004).
Paez, J G et al., “EGFR mutations in lung cancer: correlation with clinical response to gefitinib therapy,” Science, 304:1497-1500 (2004).
Pao, W et al., “KRAS mutations and primary resistance of lung adenocarcinomas to gefitinib or erlotinib,” PLoS Med., 2:e17 (2005).
Parsons, D W et al., “An integrated genomic analysis of human glioblastoma multiforme,” Science, 321(5897):1807-1812 (2008).
Perrault, S D et al., “Mediating tumor targeting efficiency of nanoparticles through design,” Nano Lett., 9(5):1909-1915 (2009).
Puvanakrishnan, P et al., “Near-infrared narrow-band imaging of gold/silica nanoshells in tumors,” J. Biomed. Opt., 14(2):024044 (2009).
Rahman, A et al., “A phase I clinical trial and pharmacokinetic evaluation of liposome-encapsulated doxorubicin,” J. Clin. Oncol., 8(6):1093-1100 (1990).
Rechberger, W et al., “Optical properties of two interacting gold nanoparticles,” Opt. Commun., 220(1-3):137-141 (2003).
Richardson, H H et al., “Experimental and theoretical studies of light-to-heat conversion and collective heating effects in metal nanoparticle solutions,” Nano Lett., 9(3):1139-1146 (2009).
Roper, D K et al., “Microscale heat transfer transduced by surface plasmon resonant gold nanoparticles,” J. Phys. Chem. C., 111(9):3636-3641 (2007).
Schwartz, J A et al., “Feasibility study of particle-assisted laser ablation of brain tumors in orthotopic canine model,” Cancer Res., 69:1659-1667 (2009).
Tanaka, T et al., “Sustained small interfering RNA delivery by mesoporous silicon particles,” Cancer Res., 70(9):3687-3696 (2010).
Tasciotti, E et al., “Mesoporous silicon particles as a multistage delivery system for imaging and therapeutic applications,” Nat. Nanotechnol., 3:151-157 (2008).
Treat, J et al., “Antitumor activity of liposome-encapsulated doxorubicin in advanced breast cancer: phase II study,” J. Natl. Cancer Inst., 82(21):1706-1710 (1990).
Winter, P M et al., “Molecular imaging of angiogenesis in nascent Vx-2 rabbit tumors using a novel alpha(nu)beta3-targeted nanoparticle and 1.5 tesla magnetic resonance imaging,” Cancer Res., 63(18):5838-5843 (2003).
Wood, L D et al., “The genomic landscapes of human breast and colorectal cancers,” Science, 318:1108-1113 (2007).
You, J et al., “Exceptionally high payload of doxorubicin in hollow gold nanospheres for near-infrared light-triggered drug release,” ACS Nano., 4:1033-1041 (2010).

All of the compositions and methods disclosed and claimed herein can be made and executed without undue experimentation in light of the present disclosure. While the compositions and methods of this invention have been described in terms of exemplary embodiments, it will be apparent to those of ordinary skill in the art that variations may be applied to the composition, methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those of ordinary skill in the art are deemed to be within the spirit, scope and concept of the invention as defined herein.

What is claimed is:

1. A composition comprising a first-stage particle that comprises porous or nanoporous silicon, and which has (i) a body, (ii) at least one surface; and (iii) at least one reservoir inside the body, such that the reservoir is adapted and configured to contain at least one second-stage particle that comprises solid gold or hollow gold.

2. The composition of claim 1, wherein the first-stage particle has a substantially spherical, a substantially discoidal, a substantially hemispherical, a substantially cylindrical, or a substantially non-spherical shape.

3. The composition of claim 1, wherein the average size of the first-stage particle is substantially from about 500 to about 3500 nm in diameter.

4. The composition of claim 1, wherein the average size of the first-stage particle is substantially from about 600 to about 3300 nm in diameter.

5. The composition of claim 1, wherein the average size of the first-stage particle is substantially from about 700 to about 3100 nm in diameter.

6. The composition of claim 1, wherein the average size of the first-stage particle is substantially from about 800 to about 3000 nm in diameter.

7. The composition of claim 1, wherein the average size of the first-stage particle is substantially from about 1000 to about 2600 nm in diameter.

8. The composition of claim 1, wherein the porous or nanoporous silicon has one or more pores, at least one of which pores is substantially about 30 to about 150 nm in diameter.

9. The composition of claim 1, characterized as a silicon nanoassembly.

10. The composition of claim 9, characterized as a gold-in-silicon nanoassembly.

11. The composition of claim 1, wherein the average size of the second-stage particle is substantially from about 5 to about 100 nm in diameter.

12. The composition of claim 1, wherein the first-stage particle is adapted and configured to selectively target one or more specific mammalian cells or cell types, or wherein the first-stage particle further comprises at least one targeting or affinity moiety operably attached thereto or therein.

13. The composition of claim 12, wherein the at least one targeting or affinity moiety is selected from the group consisting of a chemically-targeting moiety, a physically-targeting moiety, an affinity ligand targeting moiety, a geometrically-targeting moiety, and any combination thereof.

14. The composition of claim 12, wherein the first-stage particle is adapted and configured to selectively target one or more specific mammalian cells or cell types by virtue of the size, shape, dimension, or chemical composition of the body of the first-stage particle; by virtue of at least a first ionic charge on the surface of the first-stage particle; by virtue of a chemical modification, derivitization, functionalization, or liganding of one or more of the first-stage particles, or by virtue of any combination thereof.

15. The composition of claim 12, wherein a) the at least one targeting moiety comprises a chemically-targeting moiety disposed on at least a first portion of a first surface of the first-stage particle, or b) the chemically-targeting moiety comprises at least one moiety selected from a the group consisting of a dendrimer, an aptamer, an antibody, an antigen binding fragment, a peptide, a thioaptamer, a protein, an enzyme, a ligand, a cell surface receptor, a polynucleotide, a polysaccharide, a lipid, a phospholipid, an enzyme substrate, a small molecule, and any combination thereof.

16. The composition of claim 1, wherein the composition further comprises at least one additional chemotherapeutic or diagnostic agent, or a combination thereof.

17. A therapeutic or diagnostic agent delivery system comprising: the composition of claim 1, wherein at least a first population of the first-stage particles is adapted and configured to substantially retain a population of the second-stage particles within the reservoir in an amount and for a time effective to administer the delivery system to one or more cells, tissues, organs, or to the circulatory system of an animal in need thereof.

18. The therapeutic or diagnostic agent delivery system of claim 17, wherein the at least a first population of first-stage particles is adapted and configured to substantially slow, delay, or prevent release of the population of second-stage particles from the reservoir for a period of time following administration of the therapeutic or diagnostic delivery system to the one or more cells, tissues, organs, or the circulatory system of the animal.

19. The therapeutic or diagnostic agent delivery system of claim 17, wherein at least a first population of the first-stage particles and at least a first population of the second-stage particles form a mild heat, or thermal ablative nanoassembly.

20. The therapeutic or diagnostic agent delivery system of claim 17, wherein the reservoir, or the second-stage particle further comprises at least a first chemotherapeutic agent, at least a first diagnostic agent, or a combination thereof.

21. The therapeutic or diagnostic agent delivery system of claim 20, wherein the first chemotherapeutic agent is effective against one or more human cancer cells.

22. The therapeutic or diagnostic agent delivery system of claim 20, wherein the at least a first diagnostic compound comprises an imaging agent, a contrast agent, a localizing moiety, a reporter molecule, a labeled compound, or any combination thereof.

23. The therapeutic or diagnostic agent delivery system of claim 22, wherein the at least a first diagnostic compound comprises an MRI, PET, CAT, or radiographic imaging agent.

24. A method of delivering a therapeutic or a diagnostic compound to at least a first selected population of cells, one or more tissue(s), or one or more organ(s), or any combination thereof, within or about the body of a mammal in need thereof, the method comprising administering to the animal an effective amount of the therapeutic or diagnostic agent delivery system of claim 17, for a time sufficient to provide the therapeutic or diagnostic compound to the selected population of cells, the one or more tissue(s), the one or more organ(s), or the combination thereof.

25. The method of claim 24, wherein the selected population of cells comprises one or more stem cells, one or more clonogenic cells, one or more cancerous cells, one or more pre-cancerous cells, or any combination thereof.

26. The method of claim 24, wherein the one or more tissue(s) comprises at least a first tumor, cancerous lesion, pre-cancerous lesion, or abnormal growth.

27. The method of claim 24, wherein the administering includes at least the step of providing the delivery system intravascularly, subcutaneously, intrathecally, or by one or more direct injections to at least a first selected site within or about the body of the mammal.

28. A method for mild heat therapy or thermal ablation of a targeted cell or tissue in a mammal comprising administering to the mammal an effective amount of a pharmaceutical formulation that comprises the composition of claim 1, in the presence of a near-infrared (NIR) energy source in an amount and for a time sufficient to heat or thermally ablate the targeted cell or tissue.

29. A method of treating or ameliorating one or more symptoms of cancer in a mammal, comprising administering to a subject in need thereof, in the presence of a NIR energy source, an effective amount of a pharmaceutical formulation comprising the composition of claim 1, for a time sufficient to treat or ameliorate the one or more symptoms of at least a first cancer in the mammal.

30. The method of claim 28, wherein the pharmaceutical formulation further comprises a buffer, a surfactant, a polymethacrylate, a biodegradable polymer, a biodegradable polyester, an aqueous polymeric gel, a microparticle, a nanoparticle, a liposome, a nanosphere, or any combination thereof.

31. The method of claim 29, wherein the NIR energy source is adapted and configured to emit energy at one or more discreet wavelengths.

32. The method of claim 31, wherein the NIR energy source is a laser adapted and configured to emit energy at one or more discreet wavelengths of approximately 530 nm, 800 nm, or a combination thereof.
